(19) United States
(12) Patent Application Publication
(10) Pub. No.: US 2025/0161368 A1
(43) Pub. Date: May 22, 2025

(54) Cell Therapy

(71) Applicant: ASTRAZENECA AB, Södertälje (SE)

(72) Inventors: Karin Jennbacken SJÖSTRAND, Södertälje (SE); Qing-Dong WANG, Södertälje (SE); Mick FELLOWS, Cambridge (GB)

(21) Appl. No.: 18/886,624

(22) Filed: Sep. 16, 2024

(30) Foreign Application Priority Data
Sep. 18, 2023 (GB) 2314218.5

(51) Int. Cl. A61K 35/34; A61K 45/06; A61P 9/10; C12N 5/077

(52) U.S. Cl. CPC A61K 35/34; A61K 45/06; A61P 9/10; C12N 5/0657; C12N 2506/03

(57) ABSTRACT

Populations of cells for use in the treatment of heart disease and methods of making the same.

### 1 BACKGROUND

An estimated 64 million people are affected by heart failure (HF) worldwide, with the prevalence of known HF at approximately 1% to 2% of the general adult population. HF results in over one million hospitalisations annually in the United States and Europe [1]. Once diagnosed, HF patients average one hospitalisation per year [2]. The absolute number of HF hospitalisations is expected to increase in the future, potentially as much as 50% over 25 years, due to a combination of population growth, aging, and increased prevalence of comorbidities [2]. HF with low ejection fraction (LVEF)<40% accounts for approximately half of all HF cases [3,4].

The main pharmaceutical treatments for HF include beta-blockers, angiotensin converting enzyme inhibitors, an angiotensin receptor neprilysin inhibitor, SGLT2 inhibitors, mineralocorticoid receptor antagonists, as well as diuretics for fluid retention and hydralazine/nitrate therapy for selected patients [5].

Despite the existing standard of care for heart HF that includes pharmacological treatment with several available classes of medications, devices, and heart transplantation, all of which have certain limitations, there is still a clear unmet medical need as the 1-year mortality ranges from 25% to 75% for advanced HF patients [2]. Guidelines have recently been updated to incorporate, e.g., SGLT2 inhibitors and angiotensin receptor neprilysin inhibitors, but improving cardiac function and outcomes remains challenging. Numerous therapies are also in development for HF, but options for patients with advanced HF remain limited.

Foo et al. [6] described a method for generating ISL1 positive human ventricular progenitor cells from human embryonic stem cells (ESC) capable of differentiating into ventricular heart muscle in vivo (“human ventricular progenitor cells”, HVP). Populations of HVP were harvested on day 6 of differentiation, which Foo et al. identifies as the optimal differentiation window [6].

WO 2016/029122 A1 [7] described a population of human cardiac ventricular progenitor cells derived from ESC harvested on day 6 of differentiation and have been contacted with a one or more agents reactive with Jagged 1 (JAG1) and/or Frizzled 4 (FZD4).

WO 2017/172086 A1 [8] described genetic markers JAG1, FZD4, LIFR, FGFR3 and/or TNFSF9 for identifying engraftable human ventricular progenitor cells. The HVP were harvested on day 6 of differentiation from ESC.

WO 2018/100433 A1 [9] described a method of isolating human cardiac ventricular progenitor cells harvested on day 5-7 of differentiation.

WO 2019/038587 A1 described a method for isolating human cardiac ventricular progenitor cells, the method comprising contacting a culture of human cells containing cardiac progenitor cells with one or more agents reactive with neuropilin-1 (NRP1).

### 2 SUMMARY

A risk connected with pluripotent stem cell-derived cells is teratoma formation. The present disclosure, supported by data presented herein for the first time, is based on a new protocol for ventricular progenitor cell differentiation that reduces the risk of teratoma formation and provides a population of cells suitable for use as cell therapy product for administration to patients.

### 3 BRIEF DESCRIPTION OF THE DRAWINGS

FIG. 1: Cardiac progenitor differentiation protocol

Differentiation of a cell population of cells suitable for repair of cardiac ventricular tissue comprises taking a population of pluripotent stem cells in culture. On day 0 the cells are cultured in the presence of a GSK inhibitor (e.g. CHIR). On day 1 the GSK inhibitor is removed. On day 3 the cells are cultured in the presence of a WNT inhibitor. On day 5 the Wnt inhibitor is removed. On day 8 the differentiated progenitor cells are harvested and subjected to TRA-1-60 sorting to deplete TRA-1-60 expressing cells. PSC: Pluripotent stem cells; VPC: Cardiac ventricular progenitor cell; MACS: Magnetic activated cell sorting. Methods for early stage differentiation of ventricular progenitor cells (harvested at Day 6) are described in Foo et al.

FIG. 2: Gene expression of cardiac progenitor cells

PCA plot of RNAseq data from Day 0 (pluripotent cells) and Day 5 to 10 (VPCs) and Day 15 (immature cardiomyocytes).

FIG. 3: Teratoma formation by Day 6 cardiac progenitor cell populations engrafted into mouse kidneys

Out-growth on mouse kidney following injection of unsorted Day 6 cardiac progenitor cells into capsule. Left: Gross image of a teratoma; Right: Histological appearance of teratoma (HE)

FIG. 4: OCT4 and TRA-1-60 expression by Day 6 and Day 8 harvested cells

Day 6 and Day 8 harvested cell populations were TRA-1-60 sorted (depletion of TRA-1-60 expressing cells).

FIG. 5: Detecting residual pluripotent cells by qPCR, FACS and HEC assay

HEC: highly efficient culture. FACS: Fluorescence-activated cell sorting; qPCR: quantitative polymerase chain reaction.

FIG. 6: Undifferentiated marker expression in spiked Day 8 harvested differentiated VPC populations

All cell populations were derived from H9 cells. H9=human embryonic stem cell line WIC-WA09

FIG. 7: Effect of AZD6414 (Derived from H9) on Cardiac Adverse Remodelling in Mice Post-MI

Effect of VPC cells derived from H9 (AZD6414; n=15) or Vehicle (n=14) on cardiac remodelling and function after the LAD coronary artery permanent ligation in immunocompromised mice. Cells or vehicle were injected immediately after LAD ligation and cardiac function evaluated by echocardiography 24 hours, Day 28 and Day 49 post-MI. Echocardiography analysis was performed using Simpson Method. Values are presented as mean±SEM. Statistical analysis was performed using GraphPad Prism 7.1. Mixed-effect-analysis and p-value set for p<0.05. H9=human embryonic stem cell line WIC-WA09; VPC=ventricular progenitor cells; LAD=left anterior descending; MI=myocardial infarction; n=number of mice treated; SEM=standard error of the mean.

FIG. 8: HVP treatment effects on cardiac function overtime. a) Study plan schematics. b) Effects of HVP cells on left ventricle ejection fraction overtime. c) Effects of HVP cells on global longitudinal strain overtime. d) Effects of HVP cell on left ventricle stroke volume over time. e) Left ventricle wall motion score index. f) Infarct volume overtime. Mean±SEM; Vehicle, n=6; Million, n=7; Million, n=7; Million, n=6; Two Away ANOVA mixed effects followed by Dunnett's multiple comparison test. *, vs Vehicle, p-value <0.05. Day 0, 21 days post MI; Day 30, 60 and 90 post epicardial HVP cell injection.

FIG. 9: HVP cells with a mature ventricular cardiomyocyte phenotype persist in pig myocardium 3 months post-implantation.

a) HVP cells (delineated areas) in the border zone of infarcted pig myocardium express the ventricular cardiomyocyte protein MLC2v. b) Same tissue region as (a) stained with haematoxylin and eosin (H&E) confirms the absence of inflammatory infiltrates in the region of implanted cells. Black arrows indicate extent of grafts of human cells. c) High power image of HVP cells demonstrates diffuse cytoplasmic expression of the ventricular cardiomyocyte protein MLC2v (*) and presence of cross-striations (arrows) providing evidence of differentiation of HVPs cells to a mature cardiomyocyte phenotype. d) Similar pattern of immunostaining of N-cadherin (*) in human and pig cardiomyocytes provides evidence of formation of adherens junctions in HVP cells. Note multiple areas of close physical apposition of human and pig cardiomyocytes (black arrows).

### 4 DETAILED DESCRIPTION

### 4.1 Cell Population

The present disclosure relates to a cell population, wherein at least 70% of the cells in the cell population express a cardiac progenitor cell (CPC) associated marker. 1% or less of the cells in the cell population may express Octamer Transcription Factor 4 (OCT4, also known as OCT4/OCT3). The present disclosure relates to a cell population, wherein at least 70% of the cells in the cell population express a cardiac progenitor cell (CPC) associated marker and 1% or less of the cells in the cell population express Octamer Transcription Factor 4 (OCT4).

The disclosure also relates to a preparation, comprising $1% OCT4+ cells, and ≥70% ISL1+ cells.

### 4.2 Cell Population Properties

The cell population of the disclosure may comprise dissociated cells. The cell population of the disclosure may consist of dissociated cells. Cell dissociation may be achieved according to standard protocols and is a standard technique in cell culture, e.g., EDTA dissociation. The cell population may comprise of cells in suspension. The cells may consist of cells in suspension. Cells in suspension are not attached to a surface. The cell population may not be in the form of 2D-adherent monolayer. The cell population may not be in the form of a monolayer. The cell population may not be in the form of an adherent monolayer. The cell population may not be in the form of a 3D culture. The cell population may be derived from a 2D-adherent monolayer. The cell population may be cells dissociated from a 2D-adherent monolayer. The cell population may not be attached to a scaffold or extracellular matrix, e.g., Matrigel, but may be derived from cells grown on a scaffold or extracellular matrix such as Matrigel. Cells in suspension may not be attached to each other or a tissue culture surface, such as a tissue culture flask or dish. The cells in suspension may be pelleted by centrifugation. The cell population may comprise or consist of living cells. The majority of the cells in the cell population may be living cells (e.g., cells that have not been fixed). The cell population may have 50% viability. The cell population may have 55% viability. The cell population may have 60% viability. The cell population may have 65% viability. The cell population may have 70% viability. The cell population may have 75% viability. The cell population may have 80% viability. The cell population may have 85% viability. The cell population may have 90% viability. The cell population may have 95% viability. The cell population may have 96% viability. The cell population may have 97% viability. The cell population may have 98% viability. The cell population may have 99% viability. Cell viability may be measured or assessed by methods known in the art, e.g., methods detecting ATP activity.

Cell population means a population, group or collection of cells. The disclosure relates to a preparation containing or made up of cells in the form of a cell population. A cell population may comprise at least 500 cells. The cell population may comprise at least 1000 cells.

The cell population may be heterogenous. The cells in the cell population may be genetically identical. The cell population may be multipotential. The cell population may comprise or consist of adherent cells.

### 4.3 Pluripotency Associated Gene Expression

### 4.3.1 OCT4 (Pou5f1)

OCT4 (also known as OCT3/4 or OCT3) as used herein refers to the protein encoded by the gene POU5F1 (POU class 5 homeobox 1) (Gene ID: 5460, HUGO Gene Nomenclature Committee, HGNC). According to the present disclosure, less than 1% of the cells in the cell population may express OCT4. Less than 1% of the cells in the cell population may express OCT4. Less than 0.9% of the cells in the cell population may express OCT4. Less than 0.8% of the cells in the cell population may express OCT4 or OCT3/OCT4. Less than 0.7% of the cells in the cell population may express OCT4. Less than 0.6% of the cells in the cell population may express OCT4. Less than 0.5% of the cells in the cell population may express OCT4. Less than 0.4% of the cells in the cell population may express OCT4. Less than 0.3% of the cells in the cell population may express OCT4. Less than 0.2% of the cells in the cell population may express OCT4. Less than 0.1% of the cells in the cell population may express OCT4. About 0.1% of the cells in the cell population may express OCT4.

The percentage (%) of cells expressing OCT4 in the cell population may be measurable by flow cytometry. The percentage (%) of cells expressing OCT4 in the cell population may be measurable by immunofluorescence. The percentage (%) of cells expressing OCT4 in the cell population may be measurable by high-throughput immunofluorescence.

According to the disclosure, less than 1% of the cells in the cell population may express OCT4 or OCT3/OCT4 as measured by flow cytometry. Less than 1% of the cells in the cell population may express OCT4 as measured by flow cytometry. Less than 0.9% of the cells in the cell population may express OCT4 as measured by flow cytometry. Less than 0.8% of the cells in the cell population may express OCT4 as measured by flow cytometry. Less than 0.7% of the cells in the cell population may express OCT4 as measured by flow cytometry. Less than 0.6% of the cells in the cell population may express OCT4 or OCT3/OCT4 as measured by flow cytometry. Less than 0.5% of the cells in the cell population may express OCT4 or OCT3/OCT4 as measured by flow cytometry. Less than 0.4% of the cells in the cell population may express OCT4 or OCT3/OCT4 as measured by flow cytometry. Less than 0.3% of the cells in the cell population may express OCT4 or OCT3/OCT4 as measured by flow cytometry. Less than 0.2% of the cells in the cell population may express OCT4 or OCT3/OCT4 as measured by flow cytometry. Less than 0.1% of the cells in the cell population may express OCT4 or OCT3/OCT4 as measured by flow cytometry. About 0.1% of the cells in the cell population may express OCT4 or OCT4/OCT3 as measured by flow cytometry.

None of the cells in the cell population may express OCT4 or OCT3/4. None of the cells in the cell population may express OCT4 as measured by flow cytometry. Antibodies specific for OCT4 for use in immunofluorescence and flow cytometry (FACS) are commercially available, see Examples (Cat. No. 130-117-709, Miltenyi Biotec). The expression of OCT4 in the cell population may be below the limit of detection as measured by flow cytometry. The expression of OCT4 in the cell population may be below the limit of detection as measured by microarray. The expression of OCT4 in the cell population may be below the limit of detection as measured by RNA sequencing. The expression of OCT4 in the cell population may be below the limit of detection as measured by quantitative polymerase chain reaction (PCR).

The cell population may produce less than one OCT4⁺ colony per million cells, as measured by high efficiency culture (HEC) assay. The cell population may be substantially free of cells expressing OCT4. The cell population may not express OCT4 as measured by western blot. OCT4 may be human OCT4.

### 4.3.2 TRA-1-60

The disclosure also relates to a cell population, wherein 1.5% or less of the cells in the population express T-cell receptor alpha locus 1-60 (TRA-1-60). According to the disclosure, less than 1.5% of the cells in the cell population may express TRA-1-60. Less than 1.4% of the cells in the cell population may express TRA-1-60. Less than 1.3% of the cells in the cell population may express TRA-1-60. Less than 1.2% of the cells in the cell population may express TRA-1-60. Less than 1.1% of the cells in the cell population may express TRA-1-60. Less than 1% of the cells in the cell population may express TRA-1-60. Less than 0.9% of the cells in the cell population may express TRA-1-60. Less than 0.8% of the cells in the cell population may express TRA-1-60. Less than 0.7% of the cells in the cell population may express TRA-1-60. Less than 0.6% of the cells in the cell population may express TRA-1-60. Less than 0.5% of the cells in the cell population may express TRA-1-60. Less than 0.4% of the cells in the cell population may express TRA-1-60. Less than 0.3% of the cells in the cell population may express TRA-1-60. Less than 0.2% of the cells in the cell population may express TRA-1-60. The % cell expressing TRA-1-60 in the cell population may be measured by flow cytometry using an antibody specific for TRA-1-60. 1.5% or less of the cells in the population may express T-cell receptor alpha locus 1-60 (TRA-1-60).

The percentage (%) of cells expressing TRA-1-60 in the cell population may be measurable or determined by flow cytometry. The percentage (%) of cells expressing TRA-1-60 in the cell population may be measurable or determined by immunofluorescence. Antibodies specific for TRA-1-60 for use in immunofluorescence and flow cytometry (FACS) are commercially available.

The expression of TRA-1-60 in the cell population may be below the limit of detection as measured by flow cytometry. The expression of TRA-1-60 in the cell population may be below the limit of detection as measured by microarray. The expression of TRA-1-60 in the cell population may be below the limit of detection as measured by RNA sequencing. The expression of TRA-1-60 in the cell population may be below the limit of detection as measured by quantitative polymerase chain reaction (PCR).

The cell population may not express TRA-1-60 as measured by western blot. The cell population may be substantially free of cells expressing TRA-1-60. TRA-1-60 may be human TRA-1-60.

### 4.3.3 NANOG

1% or less of the cells in the population may express NANOG. Less than 0.9% of the cells in the cell population express NANOG. Less than 0.8% of the cells in the cell population express NANOG. Less than 0.7% of the cells in the cell population express NANOG. Less than 0.6% of the cells in the cell population express NANOG. Less than 0.5% of the cells in the cell population express NANOG. Less than 0.4% of the cells in the cell population express NANOG. Less than 0.3% of the cells in the cell population express NANOG. Less than 0.2% of the cells in the cell population express. The percentage (%) of cells expressing NANOG in the cell population may be measurable or determined by flow cytometry. The percentage (%) of cells expressing NANOG in the cell population may be measurable or determined by immunofluorescence. Antibodies specific for NANOG for use in immunofluorescence and flow cytometry (FACS) are commercially available.

The expression of NANOG in the cell population may be below the limit of detection as measured by flow cytometry. The expression of NANOG in the cell population may be below the limit of detection as measured by microarray. The expression of NANOG in the cell population may be below the limit of detection as measured by RNA sequencing. The expression of NANOG in the cell population may be below the limit of detection as measured by quantitative polymerase chain reaction (PCR).

The cell population may not express NANOG as measured by western blot. The cell population may be substantially free of cells expressing NANOG. NANOG may be human NANOG.

### 4.3.4 SOX2

3% or less of the cells in the population may express SOX2. Less than 2.9% of the cells in the population may express SOX2. Less than 2.8% of the cells in the population may express SOX2. Less than 2.7% of the cells in the population may express SOX2. Less than 2.6% of the cells in the population may express SOX2. Less than 2.5% of the cells in the population may express SOX2. Less than 2.4% of the cells in the population may express SOX2. Less than 2.3% of the cells in the cell population express SOX2. The expression of SOX2 in the cell population may be below the limit of detection. The percentage (%) of cells expressing SOX2 in the cell population may be measurable or determined by flow cytometry. The percentage (%) of cells expressing SOX2 in the cell population may be measurable or determined by immunofluorescence. Antibodies specific for SOX2 for use in immunofluorescence and flow cytometry (FACS) are commercially available [6].

The cell population may not express SOX2 as measured by western blot. The express of SOX2 in the cell population may be below the limit of detection as measured by flow cytometry. The express of SOX2 in the cell population may be below the limit of detection as measured by immunofluorescence. The express of SOX2 in the cell population may be below the limit of detection as measured by RNA sequencing. The express of SOX2 in the cell population may be below the limit of detection as measured by microarray. The expression of SOX2 in the cell population may be below the limit of detection as measured by quantitative polymerase chain reaction (PCR). The cell population may be substantially free of cells expressing SOX22. SOX2 may be human SOX2.

### 4.4 Cardiac Progenitor Associated Gene Expression

At least 75% of the cells in the cell population may express a cardiac progenitor cell (CPC) associated marker. At least 76% of the cells in the cell population may express a cardiac progenitor cell (CPC) associated marker. At least 77% of the cells in the cell population may express a cardiac progenitor cell (CPC) associated marker. At least 78% of the cells in the cell population may express a cardiac progenitor cell (CPC) associated marker. At least 79% of the cells in the cell population may express a cardiac progenitor cell (CPC) associated marker. At least 81% of the cells in the cell population may express a cardiac progenitor cell (CPC) associated marker. At least 80% of the cells in the cell population may express a cardiac progenitor cell (CPC) associated marker. At least 82% of the cells in the cell population may express a cardiac progenitor cell (CPC) associated marker. At least 83% of the cells in the cell population may express a cardiac progenitor cell (CPC) associated marker. At least 84% of the cells in the cell population may express a cardiac progenitor cell (CPC) associated marker. At least 85% of the cells in the cell population may express a cardiac progenitor cell (CPC) associated marker. At least 86% of the cells in the cell population may express a cardiac progenitor cell (CPC) associated marker. At least 87% of the cells in the cell population may express a cardiac progenitor cell (CPC) associated marker. At least 88% of the cells in the cell population may express a cardiac progenitor cell (CPC) associated marker. At least 89% of the cells in the cell population may express a cardiac progenitor cell (CPC) associated marker. At least 90% of the cells in the cell population may express a cardiac progenitor cell (CPC) associated marker. At least 91% of the cells in the cell population may express a cardiac progenitor cell (CPC) associated marker. At least 92% of the cells in the cell population may express a cardiac progenitor cell (CPC) associated marker. At least 93% of the cells in the cell population may express a cardiac progenitor cell (CPC) associated marker. At least 94% of the cells in the cell population may express a cardiac progenitor cell (CPC) associated marker. At least 95% of the cells in the cell population may express a cardiac progenitor cell (CPC) associated marker. At least 96% of the cells in the cell population may express a cardiac progenitor cell (CPC) associated marker. At least 97% of the cells in the cell population may express a cardiac progenitor cell (CPC) associated marker. At least 98% of the cells in the cell population may express a cardiac progenitor cell (CPC) associated marker. At least 99% of the cells in the cell population may express a cardiac progenitor cell (CPC) associated marker. About all of the cells in the cell population may express a cardiac progenitor cell (CPC) associated marker.

### 4.4.1 ISL1

The CPC associated marker may be Insulin gene enhancer protein (ISL1). At least 75% of the cells in the cell population may express ISL1. At least 76% of the cells in the cell population may express ISL1. At least 77% of the cells in the cell population may express ISL1. At least 78% of the cells in the cell population may express ISL1. At least 79% of the cells in the cell population may express ISL1. At least 80% of the cells in the cell population may express ISL1. At least 81% of the cells in the cell population may express ISL1. At least 82% of the cells in the cell population may express ISL1. At least 85% of the cells in the cell population may express ISL1. At least 83% of the cells in the cell population may express ISL1. At least 84% of the cells in the cell population may express ISL1. At least 90% of the cells in the cell population may express ISL1. At least 85% of the cells in the cell population may express ISL1. At least 86% of the cells in the cell population may express ISL1. At least 87% of the cells in the cell population may express ISL1. At least 88% of the cells in the cell population may express ISL1. At least 89% of the cells in the cell population may express ISL1. At least 90% of the cells in the cell population may express ISL1. At least 91% of the cells in the cell population may express ISL1. At least 92% of the cells in the cell population may express ISL1. At least 93% of the cells in the cell population may express ISL1. At least 94% of the cells in the cell population may express ISL1. At least 95% of the cells in the cell population may express ISL1. At least 96% of the cells in the cell population may express ISL1. At least 97% of the cells in the cell population may express ISL1. At least 98% of the cells in the cell population may express ISL1. At least 99% of the cells in the cell population may express ISL1. About all of the cells in the cell population may express ISL1.

The percentage (%) of cells expressing ISL1 in the cell population may be measurable or determined by flow cytometry. The percentage (%) of cells expressing ISL1 in the cell population may be measurable or determined by immunofluorescence. Antibodies specific for ISL1 for use in immunofluorescence and flow cytometry (FACS) are commercially available [6]. The cell population may express ISL1 as measured by western blot. ISL1 may be human ISL1.

### 4.4.2 Ventricular Progenitor Associated Markers

The cells in the cell population may express ventricular progenitor associated markers. Ventricular progenitor associated markers are described in Foo et al. [6]. The cells in the cell population may express ISL1. The cells in the cell population may express PDGFRA. The cells in the cell population may express TBX1. The cells in the cell population may express HAND1. The cells in the cell population may express TBX5. The cells in the cell population may express SMARCD3. The cell population may express Jagged-1 (JAG1). The cells in the cell population may express Frizzled-4 (FZD4). The cell population may express Fibroblast growth factor receptor 3 (FGFR3). The cell population may express Leukaemia Inhibitory Factor Receptor (LIFR). The cell population may express TNF Superfamily Member 9 (TNFSF9). The cell population may express ISL1 and LIFR. The cells in the cell population may express PDGFRA and ISL1. The cells in the cell population may express TBX1. The cells in the cell population may express HAND1 and ISL1. The cells in the cell population may express TBX5. The cells in the cell population may express SMARCD3 and ISL1. The cell population may express Jagged-1 (JAG1) and ISL1. The cells in the cell population may express Frizzled-4 (FZD4) and ISL1. The cell population may express Fibroblast growth factor receptor 3 (FGFR3) and ISL1. The cell population may express Leukaemia Inhibitory Factor Receptor (LIFR). The cell population may express TNF Superfamily Member 9 (TNFSF9). The cell population may express ISL1 and LIFR.

The percentage (%) of cells expressing PDGFRA in the cell population may be measurable or determined by flow cytometry. The percentage (%) of cells expressing PDGFRA in the cell population may be measurable or determined by immunofluorescence. Antibodies specific for PDGFRA for use in immunofluorescence and flow cytometry (FACS) are commercially available [6].

The percentage (%) of cells expressing TBX1 in the cell population may be measurable or determined by flow cytometry. The percentage (%) of cells expressing TBX1 in the cell population may be measurable or determined by immunofluorescence. Antibodies specific for TBX1 for use in immunofluorescence and flow cytometry (FACS) are commercially available [6].

The percentage (%) of cells expressing HAND1 in the cell population may be measurable or determined by flow cytometry. The percentage (%) of cells expressing HAND1 in the cell population may be measurable or determined by immunofluorescence. Antibodies specific for HAND1 for use in immunofluorescence and flow cytometry (FACS) are commercially available [6].

The percentage (%) of cells expressing SMARCD3 in the cell population may be measurable or determined by flow cytometry. The percentage (%) of cells expressing SMARCD3 in the cell population may be measurable or determined by immunofluorescence. Antibodies specific for SMARCD3 for use in immunofluorescence and flow cytometry (FACS) are commercially available [6].

At least 70% of the cells in the cell population may express LIFR and ISL1. At least 71% of the cells in the cell population may express LIFR and ISL1. At least 72% of the cells in the cell population may express LIFR and ISL1. At least 73% of the cells in the cell population may express LIFR and ISL1. At least 74% of the cells in the cell population may express LIFR and ISL1. At least 75% of the cells in the cell population may express LIFR and ISL1. At least 76% of the cells in the cell population express LIFR and ISL1. At least 77% of the cells in the cell population may express LIFR and ISL1. At least 78% of the cells in the cell population express LIFR and ISL1. At least 79% of the cells in the cell population may express LIFR and ISL1. At least 80% of the cells in the cell population may express LIFR and ISL1. At least 81% of the cells in the cell population express LIFR and ISL1. At least 82% of the cells in the cell population may express LIFR and ISL1. At least 83% of the cells in the cell population may express LIFR and ISL1. At least 84% of the cells in the cell population may express LIFR and ISL1. At least 85% of the cells in the cell population may express LIFR and ISL1. At least 85% of the cells in the cell population may express LIFR and ISL1. At least 86% of the cells in the cell population may express LIFR and ISL1. At least 87% of the cells in the cell population may express LIFR and ISL1. At least 89% of the cells in the cell population may express LIFR and ISL1. At least 70% of the cells in the cell population may express LIFR and ISL1. At least 90% of the cells in the cell population may express LIFR and ISL1. At least 91% of the cells in the cell population may express LIFR and ISL1. At least 92% of the cells in the cell population may express LIFR and ISL1. At least 93% of the cells in the cell population may express LIFR and ISL1. At least 94% of the cells in the cell population may express LIFR and ISL1. At least 95% of the cells in the cell population may express LIFR and ISL1. At least 96% of the cells in the cell population may express LIFR and ISL1. At least 97% of the cells in the cell population may express LIFR and ISL1. At least 98% of the cells in the cell population may express LIFR and ISL1. About all of the cells in the cell population may express LIFR and ISL1.

The percentage (%) of cells expressing LIFR and ISL1 in the cell population may be measurable or determined by flow cytometry. The percentage (%) of cells expressing LIFR and ISL1 in the cell population may be measurable or determined by immunofluorescence. Antibodies specific for LIFR and ISL1 for use in immunofluorescence and flow cytometry (FACS) are commercially available [6].

### 4.5 Cell Types

All of the cell in the cell population may be human cells. The cells in the cell population may comprise human cells. The cells in the cell population may be substantially all human cells. The cell population may be AZD6414. The cell population may comprise AZD6414. The cell population may comprise cardiac progenitor cells. The cell population may comprise human ventricular progenitor cells (HVP). The cell population may comprise ventricular progenitor cells (VPC). The cell population may comprise hPSCs-pan mesoderm-cardiac mesoderm-islet ventricular progenitor cells.

80% or more of the cells in the cell population may be HVP. 81% or more of the cells in the cell population may be HVP. 82% or more of the cells in the cell population may be HVP. 83% or more of the cells in the cell population may be HVP. 84% or more of the cells in the cell population may be HVP. 85% or more of the cells in the cell population may be HVP. 86% or more of the cells in the cell population may be HVP. 87% or more of the cells in the cell population may be HVP. 88% or more of the cells in the cell population may be HVP. 89% or more of the cells in the cell population may be HVP. 90% or more of the cells in the cell population may be HVP. 91% or more of the cells in the cell population may be HVP. 92% or more of the cells in the cell population may be HVP. 93% or more of the cells in the cell population may be HVP. 94% or more of the cells in the cell population may be HVP. 95% or more of the cells in the cell population may be HVP. 96% or more of the cells in the cell population may be HVP. 97% or more of the cells in the cell population may be HVP. 98% or more of the cells in the cell population may be HVP. 99% or more of the cells in the cell population may be HVP. About all of the cells in the cell population may be HVP.

80% or more of the cells in the cell population may be VPC. 81% or more of the cells in the cell population may be VPC. 82% or more of the cells in the cell population may be VPC. 83% or more of the cells in the cell population may be VPC. 84% or more of the cells in the cell population may be HVP. 85% or more of the cells in the cell population may be VPC. 86% or more of the cells in the cell population may be VPC. 87% or more of the cells in the cell population may be VPC. 88% or more of the cells in the cell population may be VPC. 89% or more of the cells in the cell population may be HVP. 90% or more of the cells in the cell population may be VPC. 91% or more of the cells in the cell population may be VPC. 92% or more of the cells in the cell population may be VPC. 93% or more of the cells in the cell population may be VPC. 94% or more of the cells in the cell population may be HVP. 95% or more of the cells in the cell population may be VPC. 96% or more of the cells in the cell population may be VPC. 97% or more of the cells in the cell population may be VPC. 98% or more of the cells in the cell population may be VPC. 99% or more of the cells in the cell population may be VPC. About all of the cells in the cell population may be VPC.

5% or less of the cells in the cell population may be stem cells. 4% or less of the cells in the cell population may be stem cells. 3% or less of the cells in the cell population may be stem cells. 2% or less of the cells in the cell population may be stem cells. 1% or less of the cells in the cell population may be stem cells. There may be no stem cells in the cell population.

5% or less of the cells in the cell population may pluripotent. 4% or less of the cells in the cell population may be pluripotent. 3 or less of the cells in the cell population may be pluripotent. 2% or less of the cells in the cell population may be pluripotent. 1% or less of the cells in the cell population may be pluripotent. There more be no pluripotent cells in the cell population.

10% or less of the cells in the cell population may be fully differentiated. 9% or less of the cells in the cell population may be fully differentiated. 8% or less of the cells in the cell population may be fully differentiated. 7% or less of the cells in the cell population may be fully differentiated. 6% or less of the cells in the cell population may be fully differentiated. 5% or less of the cells in the cell population may be fully differentiated. 4% or less of the cells in the cell population may be fully differentiated. 3% or less of the cells in the cell population may be fully differentiated. 2% or less of the cells in the cell population may be fully differentiated. 1% or less of the cells in the cell population may be fully differentiated. None of the cells in the cell population may be fully differentiated.

10% or less of the cells in the cell population may be terminally differentiated. 9% or less of the cells in the cell population may be fully differentiated. 8% or less of the cells in the cell population may be fully differentiated. 7% or less of the cells in the cell population may be terminally differentiated. 6% or less of the cells in the cell population may be terminally differentiated. 5% or less of the cells in the cell population may be terminally differentiated. 4% or less of the cells in the cell population may be terminally differentiated. 3% or less of the cells in the cell population may be terminally differentiated. 2% or less of the cells in the cell population may be terminally differentiated. 1% or less of the cells in the cell population may be terminally differentiated. None of the cells in the cell population may be terminally differentiated.

The cell population may comprise or consist of somatic cells. The cell population may comprise or consist of adult cells. The cell population may not comprise cardiac atrial progenitor cells.

The ventricular progenitor cells may be committed to the cardiac lineage. The VPCs may have the capacity to differentiate into all three cardiac lineage cells (cardiac muscle cells, endothelial cells and smooth muscle cells). A culture of human cardiac progenitor cells can be obtained by, for example, culturing stem cells under conditions that bias the stem cells toward differentiation to the cardiac lineage.

### 4.6 Function

The cell population may be committed to the cardiac lineage. The cells in the cell population may be committed to the cardiac ventricular lineage. The cells in the cell population may only be further differentiated into cardiac tissue or ventricular cardiac tissue.

Following delivery to the ventricular wall of a subject the cell population may not form a teratoma. The subject may be human. The subject may be a pig. The subject may be a mouse. The subject may be a minipig. The cell population may comprise or consist of cells that have been engineered to be hypoimmunogenic. The cell population may comprise or consist of cells that have been engineered to be hypo allogenic. The cell population may comprise cells that can differentiate in vitro into cardiomyocytes. The cell population may comprise cells that can differentiate in vitro into beating cardiomyocytes.

Following differentiation in vitro for at least 15 days, at least 70% of the cells in the cell population may express cardiac troponin T. Following delivery to the heart of a subject, the cell population may differentiate into heart tissue. Following delivery to the ventricular wall of a subject, the cell population may differentiate into ventricular wall tissue. Following delivery to the ventricular wall of a subject, the cell population may differentiate into beating heart muscle. Following delivery to a damaged heart of a subject, the cell population may repair damaged heart tissue. The cell population may be suitable for repairing damaged heart tissue. The subject may be human. The subject may be a pig. The subject may be a mouse. The subject may be a minipig. The cell population may be suitable for repairing damaged myocardium. Following delivery of the cell population to the heart of a subject, the cell population may forma a vascularized, electrically responsive ventricular muscle patch that secretes an extracellular matrix. Following delivery of the cell population to the tissue, e.g., kidney or heart tissue, the cell population may be capable of producing laminin. Following delivery of the cell population to the tissue, e.g., kidney or heart tissue, the cell population may be capable of producing cardiac laminin.

### 4.7 Disclaimers

The cell population may not be sorted for Jagged 1 (JAG1) expression. The cell population may not be sorted for Fizzled 4 expression. The cell population may not be sorted for neurophilin-1 (NRP1) expression. The cell population may not have been contacted with an agent that binds to Jagged 1. The cell population may not have been contacted with an agent that binds to Fizzled 4. The cell population may not have been contacted with an agent that binds to NRP1. The cells in the cell population may not have been isolated from post-natal myocardium.

The cell population may not have been derived from cells isolated from heart tissue.

### 4.8 Cardiac Muscle Associated Gene Expression

The cells in the cell population may not express cardiac muscle associated markers. Less than 10% of the cells in the cell population may express a cardiac muscle associated markers. Less than 9% of the cells in the cell population may express a cardiac muscle associated markers. Less than 8% of the cells in the cell population may express a cardiac muscle associated markers. Less than 7% of the cells in the cell population may express a cardiac muscle associated markers. Less than 6% of the cells in the cell population may express a cardiac muscle associated markers. Less than 5% of the cells in the cell population may express a cardiac muscle associated markers. Less than 4% of the cells in the cell population may express a cardiac muscle associated markers. Less than 3% of the cells in the cell population may express a cardiac muscle associated markers. Less than 2% of the cells in the cell population may express a cardiac muscle associated markers. Less than 1% of the cells in the cell population may express a cardiac muscle associated markers. The cardiac muscle associated markers may comprise TNNT2. The cardiac muscle associated markers may comprise TNNC1. The cardiac muscle associated markers may comprise MYL2. The cardiac muscle associated markers may comprise MYL7. The cardiac muscle associated markers may comprise MYH6. The cardiac muscle associated markers may comprise IRX4. The cardiac muscle associated markers may comprise SSEA-3.

### 4.8.1.1 TNNT2

Less than 10% of the cells in the cell population may express TNNT2. Less than 9% of the cells in the cell population may express TNNT2. Less than 8% of the cells in the cell population may express TNNT2. Less than 7% of the cells in the cell population may express TNNT2. Less than 6% of the cells in the cell population may express TNNT2. Less than 5% of the cells in the cell population may express TNNT2. Less than 4% of the cells in the cell population may express TNNT2. Less than 3% of the cells in the cell population may express TNNT2. Less than 2% of the cells in the cell population may express TNNT2. Less than 1% of the cells in the cell population may express TNNT2. Less than 0.9% of the cells in the cell population may express TNNT2. Less than 0.8% of the cells in the cell population may express TNNT2. Less than 0.7% of the cells in the cell population may express TNNT2. Less than 0.6% of the cells in the cell population may express TNNT2. Less than 0.4% of the cells in the cell population may express TNNT2. Less than 0.3% of the cells in the cell population may express TNNT2. Less than 0.2% of the cells in the cell population may express TNNT2. Less than 0.1% of the cells in the cell population may express TNNT2.

The expression of TNNT2 in the cell population may be below the limit of detection. The percentage (%) of cells expressing TNNT2 in the cell population may be measurable or determined by flow cytometry. The percentage (%) of cells expressing TNNT2 in the cell population may be measurable or determined by immunofluorescence. Antibodies specific for TNNT2 for use in immunofluorescence and flow cytometry (FACS) are commercially available [6].

The cell population may not express TNNT2 as measured by western blot. The express of TNNT2 in the cell population may be below the limit of detection as measured by flow cytometry. The express of TNNT2 in the cell population may be below the limit of detection as measured by immunofluorescence. The express of TNNT2 in the cell population may be below the limit of detection as measured by RNA sequencing. The express of TNNT2 in the cell population may be below the limit of detection as measured by microarray. The expression of TNNT2 in the cell population may be below the limit of detection as measured by quantitative polymerase chain reaction (PCR). The cell population may be substantially free of cells expressing TNNT2. TNNT2 may be human TNNT2.

### 4.8.1.2 TNNC1

Less than 10% of the cells in the cell population may express TNNC1. Less than 9% of the cells in the cell population may express TNNC1. Less than 8% of the cells in the cell population may express TNNC1. Less than 7% of the cells in the cell population may express TNNC1. Less than 6% of the cells in the cell population may express TNNC1. Less than 5% of the cells in the cell population may express TNNC1. Less than 4% of the cells in the cell population may express TNNC1. Less than 3% of the cells in the cell population may express TNNC1. Less than 2% of the cells in the cell population may express TNNC1. Less than 1% of the cells in the cell population may express TNNC1. Less than 0.9% of the cells in the cell population may express TNNC1. Less than 0.8% of the cells in the cell population may express TNNC1. Less than 0.7% of the cells in the cell population may express TNNC1. Less than 0.6% of the cells in the cell population may express TNNC1. Less than 0.4% of the cells in the cell population may express TNNC1. Less than 0.3% of the cells in the cell population may express TNNC1. Less than 0.2% of the cells in the cell population may express TNNC1. Less than 0.1% of the cells in the cell population may express TNNC1. The cell population may be substantially free of cells expressing TNNC1.

The expression of TNNC1 in the cell population may be below the limit of detection. The percentage (%) of cells expressing TNNC 1 in the cell population may be measurable or determined by flow cytometry. The percentage (%) of cells expressing TNNC1 in the cell population may be measurable or determined by immunofluorescence. Antibodies specific for TNNC1 for use in immunofluorescence and flow cytometry (FACS) are commercially available [6].

The cell population may not express TNNC1 as measured by western blot. The express of TNNC1 in the cell population may be below the limit of detection as measured by flow cytometry. The express of TNNC1 in the cell population may be below the limit of detection as measured by immunofluorescence. The express of TNNC1 in the cell population may be below the limit of detection as measured by RNA sequencing. The express of TNNC1 in the cell population may be below the limit of detection as measured by microarray. The expression of TNNC1 in the cell population may be below the limit of detection as measured by quantitative polymerase chain reaction (PCR). TNNC1 may be human TNNC1.

### 4.8.1.3 MYL2

Less than 10% of the cells in the cell population may express MYL2. Less than 9% of the cells in the cell population may express MYL2. Less than 8% of the cells in the cell population may express MYL2. Less than 7% of the cells in the cell population may express MYL2. Less than 6% of the cells in the cell population may express MYL2. Less than 5% of the cells in the cell population may express MYL2. Less than 4% of the cells in the cell population may express MYL2. Less than 3% of the cells in the cell population may express MYL2. Less than 2% of the cells in the cell population may express MYL2. Less than 1% of the cells in the cell population may express MYL2. Less than 0.9% of the cells in the cell population may express MYL2. Less than 0.8% of the cells in the cell population may express MYL2. Less than 0.7% of the cells in the cell population may express MYL2. Less than 0.6% of the cells in the cell population may express MYL2. Less than 0.4% of the cells in the cell population may express MYL2. Less than 0.3% of the cells in the cell population may express MYL2. Less than 0.2% of the cells in the cell population may express MYL2. Less than 0.1% of the cells in the cell population may express MYL2.

The expression of MYL2 in the cell population may be below the limit of detection. The percentage (%) of cells expressing MYL2 in the cell population may be measurable or determined by flow cytometry. The percentage (%) of cells expressing MYL2 in the cell population may be measurable or determined by immunofluorescence. Antibodies specific for MYL2 for use in immunofluorescence and flow cytometry (FACS) are commercially available [6]

The cell population may not express MYL2 as measured by western blot. The express of MYL2 in the cell population may be below the limit of detection as measured by flow cytometry. The express of MYL2 in the cell population may be below the limit of detection as measured by immunofluorescence. The express of MYL2 in the cell population may be below the limit of detection as measured by RNA sequencing. The express of MYL2 in the cell population may be below the limit of detection as measured by microarray. The expression of MYL2 in the cell population may be below the limit of detection as measured by quantitative polymerase chain reaction (PCR).

The cell population may be substantially free of cells expressing MYL2. MYL2 may be human MYL2.

### 4.8.1.4 MYL7

Less than 10% of the cells in the cell population may express MYL7. Less than 9% of the cells in the cell population may express MYL7. Less than 8% of the cells in the cell population may express MYL7. Less than 7% of the cells in the cell population may express MYL7. Less than 6% of the cells in the cell population may express MYL7. Less than 5% of the cells in the cell population may express MYL7. Less than 4% of the cells in the cell population may express MYL7. Less than 3% of the cells in the cell population may express MYL7. Less than 2% of the cells in the cell population may express MYL7. Less than 1% of the cells in the cell population may express MYL7. Less than 0.9% of the cells in the cell population may express MYL7. Less than 0.8% of the cells in the cell population may express MYL7. Less than 0.7% of the cells in the cell population may express MYL7. Less than 0.6% of the cells in the cell population may express MYL7. Less than 0.4% of the cells in the cell population may express MYL7. Less than 0.3% of the cells in the cell population may express MYL7. Less than 0.2% of the cells in the cell population may express MYL7. Less than 0.1% of the cells in the cell population may express MYL7.

The expression of MYL7 in the cell population may be below the limit of detection. The percentage (%) of cells expressing MYL7 in the cell population may be measurable or determined by flow cytometry. The percentage (%) of cells expressing MYL7 in the cell population may be measurable or determined by immunofluorescence. Antibodies specific for MYL7 for use in immunofluorescence and flow cytometry (FACS) are commercially available [6]

The cell population may not express MYL7 as measured by western blot. The express of MYL7 in the cell population may be below the limit of detection as measured by flow cytometry. The express of MYL7 in the cell population may be below the limit of detection as measured by immunofluorescence. The express of MYL7 in the cell population may be below the limit of detection as measured by RNA sequencing. The express of MYL7 in the cell population may be below the limit of detection as measured by microarray. The expression of MYL7 in the cell population may be below the limit of detection as measured by quantitative polymerase chain reaction (PCR).

The cell population may be substantially free of cells expressing MYL7. MYL7 may be human MYL7.

### 4.8.1.5 MYH6

Less than 10% of the cells in the cell population may express MYH6. Less than 9% of the cells in the cell population may express MYH6. Less than 8% of the cells in the cell population may express MYH6. Less than 7% of the cells in the cell population may express MYH6. Less than 6% of the cells in the cell population may express MYH6. Less than 5% of the cells in the cell population may express MYH6. Less than 4% of the cells in the cell population may express MYH6. Less than 3% of the cells in the cell population may express MYH6. Less than 2% of the cells in the cell population may express MYH6. Less than 1% of the cells in the cell population may express MYH6. Less than 0.9% of the cells in the cell population may express MYH6. Less than 0.8% of the cells in the cell population may express MYH6. Less than 0.7% of the cells in the cell population may express MYH6. Less than 0.6% of the cells in the cell population may express MYH6. Less than 0.4% of the cells in the cell population may express MYH6. Less than 0.3% of the cells in the cell population may express MYH6. Less than 0.2% of the cells in the cell population may express MYH6. Less than 0.1% of the cells in the cell population may express MYH6.

The expression of MYH6 in the cell population may be below the limit of detection. The percentage (%) of cells expressing MYH6 in the cell population may be measurable or determined by flow cytometry. The percentage (%) of cells expressing MYH6 in the cell population may be measurable or determined by immunofluorescence. Antibodies specific for MYH6 for use in immunofluorescence and flow cytometry (FACS) are commercially available [6].

The cell population may not express MYH6 as measured by western blot. The express of MYH6 in the cell population may be below the limit of detection as measured by flow cytometry. The express of MYH6 in the cell population may be below the limit of detection as measured by immunofluorescence. The express of MYH6 in the cell population may be below the limit of detection as measured by RNA sequencing. The express of MYH6 in the cell population may be below the limit of detection as measured by microarray. The expression of MYH6 in the cell population may be below the limit of detection as measured by quantitative polymerase chain reaction (PCR).

The cell population may be substantially free of cells expressing MYH6. MYH6 may be human MYH6.

### 4.8.1.6 IRX4

Less than 10% of the cells in the cell population may express IRX4. Less than 9% of the cells in the cell population may express IRX4. Less than 8% of the cells in the cell population may express IRX4. Less than 7% of the cells in the cell population may express IRX4. Less than 6% of the cells in the cell population may express IRX4. Less than 5% of the cells in the cell population may express IRX4. Less than 4% of the cells in the cell population may express IRX4. Less than 3% of the cells in the cell population may express IRX4. Less than 2% of the cells in the cell population may express IRX4. Less than 1% of the cells in the cell population may express IRX4. Less than 0.9% of the cells in the cell population may express IRX4. Less than 0.8% of the cells in the cell population may express IRX4. Less than 0.7% of the cells in the cell population may express IRX4. Less than 0.6% of the cells in the cell population may express IRX4. Less than 0.4% of the cells in the cell population may express IRX4. Less than 0.3% of the cells in the cell population may express IRX4. Less than 0.2% of the cells in the cell population may express IRX4. Less than 0.1% of the cells in the cell population may express IRX4.

The expression of IRX4 in the cell population may be below the limit of detection. The percentage (%) of cells expressing IRX4 in the cell population may be measurable or determined by flow cytometry. The percentage (%) of cells expressing IRX4 in the cell population may be measurable or determined by immunofluorescence. Antibodies specific for IRX4 for use in immunofluorescence and flow cytometry (FACS) are commercially available [6].

The cell population may not express IRX4 as measured by western blot. The express of IRX4 in the cell population may be below the limit of detection as measured by flow cytometry. The express of IRX4 in the cell population may be below the limit of detection as measured by immunofluorescence. The express of IRX4 in the cell population may be below the limit of detection as measured by RNA sequencing. The express of IRX4 in the cell population may be below the limit of detection as measured by microarray. The expression of IRX4 in the cell population may be below the limit of detection as measured by quantitative polymerase chain reaction (PCR).

The cell population may be substantially free of cells expressing IRX4. IRX4 may be human IRX4.

### 4.8.1.7 SSEA-3

Less than 10% of the cells in the cell population may express SSEA-3. Less than 9% of the cells in the cell population may express SSEA-3. Less than 8% of the cells in the cell population may express SSEA-3. Less than 7% of the cells in the cell population may express SSEA-3. Less than 6% of the cells in the cell population may express SSEA-3. Less than 5% of the cells in the cell population may express SSEA-3. Less than 4% of the cells in the cell population may express SSEA-3. Less than 3% of the cells in the cell population may express SSEA-3. Less than 2% of the cells in the cell population may express SSEA-3. Less than 1% of the cells in the cell population may express SSEA-3. Less than 0.9% of the cells in the cell population may express SSEA-3. Less than 0.8% of the cells in the cell population may express SSEA-3. Less than 0.7% of the cells in the cell population may express SSEA-3. Less than 0.6% of the cells in the cell population may express SSEA-3. Less than 0.4% of the cells in the cell population may express SSEA-3. Less than 0.3% of the cells in the cell population may express SSEA-3. Less than 0.2% of the cells in the cell population may express SSEA-3. Less than 0.1% of the cells in the cell population may express SSEA-3.

The expression of SSEA-3 in the cell population may be below the limit of detection. The percentage (%) of cells expressing SSEA-3 in the cell population may be measurable or determined by flow cytometry. The percentage (%) of cells expressing SSEA-3 in the cell population may be measurable or determined by immunofluorescence. Antibodies specific for SSEA-3 for use in immunofluorescence and flow cytometry (FACS) are commercially available [6].

The cell population may not express SSEA-3 as measured by western blot. The express of SSEA-3 in the cell population may be below the limit of detection as measured by flow cytometry. The express of SSEA-3 in the cell population may be below the limit of detection as measured by immunofluorescence. The express of SSEA-3 in the cell population may be below the limit of detection as measured by RNA sequencing. The express of SSEA-3 in the cell population may be below the limit of detection as measured by microarray. The expression of SSEA-3 in the cell population may be below the limit of detection as measured by quantitative polymerase chain reaction (PCR). The cell population may be substantially free of cells expressing SSEA-3. SSEA-3 may be human SSEA-3.

4.9 Cell source

The cell population may comprise or consists of cells derived from stem cells. The stem cells may be embryonic stem cells (ESCs). The cell population may comprise or consist of cells derived from a stem cell line. The stem cell line may be H9 cells. The GMP MCB H9 is derived from the source cell line WiCell WA (WiCell Research Institute) 09, also referred to as H9. The cell population may comprise or consist of cells derived from induced pluripotent stem cells (IPSCs). The cell population may not comprise any cells derived from totipotent cells.

The cell population may be from cells comprising stem cells. The cell population may be derived from stem cells. The cell population may be derived from pluripotent cells. The cell population may be derived from cells comprising embryonic stem cells. The cell population may be derived from cells comprising induced pluripotent stem cells. The cell population may be derived from cells comprising mesoderm cells. The cells may be derived from cells comprising intermediate mesoderm cells.

### 4.10 Non-Natural Products

The cell population may be is derived from a cell or cells that have been expanded ex vivo. The cell population may be derived from cell or cells isolated from a subject. The cell population may be artificial. The cell population may not exist in nature. The cell population may comprise a cell or cells that have been genetically modified or gene edited. The cell population may be isolated from the body. The cell population may be comprised in a pharmaceutical composition. The cell population may be isolated from the body and comprised in a pharmaceutical composition.

The cells in the cell population may be for example autologous, allogeneic or xenogeneic.

The cell population may be derived from a cell or cells that have been expanded ex vivo. The cell population may be derived from a cell or cells isolated from a subject. The cell population may be artificial or consist or comprise of cells that are artificial. The cell population may not exist in nature or consist or comprise of cells that do not exist in nature. The cell population may comprise cells that have been modified, e.g., by CRISPR gene editing or genetic modification. The cell population may be an isolated cell population. The cell population may comprise an engineered cell. The cell population may comprise a cell or cells that have been modified or engineered in vitro or ex vivo.

The cell population of any preceding claim, wherein the cell population comprises cells that have been modified or engineered ex vivo. The cell population wherein in the cell population is isolated from the body and is comprised in a pharmaceutical composition.

The disclosure also relates to a method of making a pharmaceutical composition comprising combining the cell population of the disclosure with a pharmaceutical excipient. The disclosure also relates to a method of making a pharmaceutical composition comprising combining a cell population of with a pharmaceutical excipient, wherein at least 70% of the cells in the cell population express a cardiac progenitor cell (CPC) associated marker, and 1% or less of the cells in the cell population express Octamer Transcription Factor 4 (OCT4). The disclosure also relates to a method of making a pharmaceutical composition comprising combining a cell population of with a pharmaceutical excipient, wherein at least 90% of the cells in the cell population express a cardiac progenitor cell (CPC) associated marker, and 1% or less of the cells in the cell population express Octamer Transcription Factor 4 (OCT4). The cardiac progenitor cell associated marker may be ISL1.

The disclosure also relates to the use of an isolated cell population, wherein at least 70% of the cells in the population express a cardiac progenitor cell (CPC) associated marker, and 1% or less of the cells in the population express Octamer Transcription Factor 4 (OCT4). The disclosure also relates to the use of an isolated cell population, wherein at least 90% of the cells in the population express a cardiac progenitor cell (CPC) associated marker, and 1% or less of the cells in the population express Octamer Transcription Factor 4 (OCT4). The cardiac progenitor cell associated marker may be ISL1.

The disclosure also relates to a composition or preparation comprising the cell population of the disclosure. The preparation may be non-natural. The disclosure also relates to a cryopreservation comprising the cell population of the disclosure. The disclosure also relates to an in vitro cell culture comprising the cell population of the disclosure.

### 4.11 Process

The disclosure also relates to a method of producing a cell population of the disclosure, the method comprising harvesting cells from a cell culture, wherein the harvested cells are derived from stem cells cultured in conditions suitable for cardiac myogenesis for 8 to 12 days. The disclosure also relates to a method of producing a cell population of the disclosure, the method comprising harvesting cells from a cell culture, wherein the harvested cells are derived from stem cells cultured in conditions suitable for cardiac myogenesis for 8 to 10 days. The conditions for cardiac myogenesis may be conditions suitable for differentiation of stem cells into cardiac ventricular cells. The disclosure also relates to a method of producing a cell population of the disclosure, the method comprising harvesting cells from a cell culture, wherein the harvested cells are derived from stem cells cultured in conditions suitable for cardiac myogenesis for about 8 days. The disclosure also relates to a method of producing a cell population of the disclosure, the method comprising harvesting cells from a cell culture, wherein the harvested cells are derived from stem cells cultured in conditions suitable for cardiac myogenesis for about 9 days. The disclosure also relates to a method of producing a cell population of the disclosure, the method comprising harvesting cells from a cell culture, wherein the harvested cells are derived from stem cells cultured in conditions suitable for cardiac myogenesis for 10 days.

A method of producing a cell population comprising cardiac ventricular progenitor cells, the method comprising harvesting a population of cells, wherein the harvested cells are derived from stem cells cultured in conditions suitable for cardiac myogenesis for 8 to 12 days. The conditions for cardiac myogenesis may be conditions suitable for differentiation of stem cells into cardiac ventricular progenitor cells.

The conditions suitable for cardiac myogenesis may comprise:
a. at day 0, activating Wnt/Wnt/β-catenin signalling in the culture,
b. at day 3-5, inhibiting Wnt/β-catenin signalling in the culture to generate a population of ventricular progenitor cells (VPCs).

Wnt/Wnt/β-catenin signalling may be activated by a GSK3 inhibitor. An example GSK3 inhibitor is CHIR-98014. Wnt/β-catenin signalling may be inhibited by a Wnt inhibitor. The Wnt inhibitor may be Wnt-C59.

The method of producing the cell population may comprise depleting the harvested cells of pluripotent cells to produce a purified population of VPCs. Depleting the harvested cells of pluripotent cells may comprise depleting the harvested cells of cells expressing a marker of pluripotency. Depleting the harvested population of VPCs of pluripotent cells may comprise depleting the population of cells expressing TRA-1-60. Depleting the harvested population of VPCs of pluripotent cells may comprise depleting the population of cells expressing TRA-1-60 by magnetic-activated cell sorting. The stem cells used in the method may be induced pluripotent cells, or embryonic stem cells. In the cell population produced by the method, at least 70% of the cells in the cell population may express a cardiac progenitor cell (CPC) associated marker, and 1% or less of the cells in the cell population may express Octamer Transcription Factor 4 (OCT4).

Methods of the disclosure may comprise various manufacturing steps that require manipulation of cells, for example, steps of seeding, feeding, dissociating an adherent culture, or washing.

### 4.12 Process Disclaimers

The disclosure also relates to a method for producing the cell populations of the disclosure, wherein the method does not comprise sorting the cells for NRP1 expression. The method may not comprise sorting the cells for Jagged 1 expression. The method may not comprise sorting the cells for Fizzled 4 expression. The method may not comprise the use of an agent that binds neuropilin-1 (NRP1). The method may not comprise the use of an agent that binds Jagged 1. The method may not comprise the use of an agent that binds Frizzled 4. The method may not comprise the seeding the cells onto a substrate including LN-521, LN-51 1 or LN-221. The method of the disclosure may therefore not comprise the unnecessary steps of sorting for markers selected form Jagged, Frizzled or NRP1. As demonstrated in the Examples, the method for producing a cell population suitable for use a cell therapy product in the treatment of e.g., heart failure, is a method requiring at least 8 days of differentiation. The cells may then be sorted to deplete TRA-1-60 expressing cells, and do not need sorting for NRP1, Jagged or Frizzled expression. A cell population expressing ISL1, and comprising 1% of less of cells expressing OCT4 lacks the capacity for pluripotency (e.g., as measured by HEC assay), and is therefore suitable for use as a cell therapy product.

### 4.13 Product-by-Process

The disclosure also relates to a cell population, wherein the cell population is produced by a method comprising harvesting a population of cells, wherein the harvested cells are derived from stem cells and have been cultured in conditions suitable for cardiac myogenesis for 8 to 12 days. The conditions for cardiac myogenesis may be conditions suitable for differentiation of stem cells into cardiac ventricular progenitor cells.

The disclosure also relates to a cell population comprising cardiac ventricular progenitor cells, wherein the cell population is produced by a method comprising harvesting cells from a cell culture, wherein the harvested cells are derived from stem cells and wherein, prior to harvesting, have been cultured in conditions suitable for cardiac myogenesis for 8 to 12 days. The conditions for cardiac myogenesis may be conditions suitable for differentiation of stem cells into cardiac ventricular progenitor cells.

The conditions suitable for cardiac myogenesis may comprise:
a. at day 0, activating Wnt/Wnt/β-catenin signalling in the culture,
b. at day 3-5, inhibiting Wnt/β-catenin signalling in the culture to generate a population of ventricular progenitor cells (VPC).

Wnt/Wnt/β-catenin signalling may be activated by a GSK3 inhibitor. An example GSK3 inhibitor is CHIR-98014. Wnt/β-catenin signalling may be inhibited by a Wnt inhibitor. The Wnt inhibitor may be Wnt-C59.

The method of producing the cell population may comprise depleting the harvested cells of pluripotent cells to produce a purified population of VPCs. Depleting the harvested cells of pluripotent cells may comprise depleting the harvested cells of cells expressing a marker of pluripotency Depleting the harvested population of CPCs of pluripotent cells comprises depleting the population of cells expressing TRA-1-60. Depleting the harvested population of CPCs of pluripotent cells comprises depleting the population of cells expressing TRA-1-60 by magnetic-activated cell sorting. The stem cells used in the method may be induced pluripotent cells, or embryonic stem cells. In the cell population produced by the method, at least 70% of the cells in the cell population may express a cardiac progenitor cell (CPC) associated marker, and 1% or less of the cells in the cell population may express Octamer Transcription Factor 4 (OCT4).

Cells may be harvested according to procedures known in the art, such as trypsin dissociation. Once harvested the cells will no longer be in a 2D monolayer culture but may be in suspension (e.g., in cell culture media) or in the form of a cell pellet. The harvested cells may be contained in a vial or tube.

### 4.14 Method of Treating

The disclosure also relates to methods of treating or prevent a disease in a subject, the method comprising administering to a subject in need thereof an effective amount of the cell population of the disclosure, wherein at least 70% of the cells in the cell population express a cardiac progenitor cell (CPC) associated marker and 1% or less of the cells in the cell population express Octamer Transcription Factor 4 (OCT4) or OCT4/OCT3. The cell population may be allogeneic or allogenic with respect to the subject. The cell population may comprise or consist of cells that are be allogeneic or allogenic with respect to the subject. The cell population may be administered with immunosuppressive therapy. The cell population may comprise or consist of cells derived from an autologous cell with respect to the subject. The cell population may be administered to the subject without immunosuppressive therapy. The cell population may comprise cells that are derived from a cell or cells that were not isolated from the subject. The cell population may comprise cells derived from a cell or cells isolated from the subject. The disease may be heart failure. The subject may be a human. The subject may be an adult human. The subject may be suffering from heart disease. The disease may be heart failure. The disease may be chronic ischemic cardiomyopathy. The subject may be suffering from chronic ischemic cardiomyopathy. The disease may be myocardial infarction. The subject may be suffering from myocardial infarction. The subject may be on immunosuppressive therapy. The disclosure also relates to the use of the cell population to prevent or treat a disease associated with loss and myocardium and/or fibrotic scarring.

The disclosure also relates to a method of treating or prevent a disease in a subject, the method comprising: a) producing in vitro a cell population according to the disclosure, and b) administering the cell population into the subject, wherein at least 70% of the cells in the cell population express a cardiac progenitor cell (CPC) associated marker and 1% or less of the cells in the cell population express Octamer Transcription Factor 4 (OCT4) or OCT4/OCT3.

### 4.15 Pharmaceutical Composition

The disclosure also relates to a pharmaceutical composition comprising cell population of the disclosure, and a pharmaceutical excipient. The disclosure also relates to a pharmaceutical composition comprising cell population and a pharmaceutical excipient, wherein at least 70% of the cells in the cell population express a cardiac progenitor cell (CPC) associated marker and 1% or less of the cells in the cell population express Octamer Transcription Factor 4 (OCT4) or OCT4/OCT3.

The pharmaceutical composition may comprise freezing media.

### 4.16 Container

The disclosure also relates to a container comprising the pharmaceutical composition of the disclosure, or the cell population of the disclosure, wherein at least 70% of the cells in the cell population express a cardiac progenitor cell (CPC) associated marker and 1% or less of the cells in the cell population express Octamer Transcription Factor 4 (OCT4) or OCT4/OCT3. The container may be a storage vial, cryovial, injection device or syringe.

### 4.17 EPC 2000

The disclosure also relates to a pharmaceutical composition for use in a method of treating or preventing a disease in a subject in need thereof, wherein the pharmaceutical composition comprises the cell population of the disclosure. The disclosure also relates to a pharmaceutical composition for use in a method of treating or preventing a disease in a subject wherein the method of treatment or preventing a disease comprises administering the pharmaceutical composition to the subject, wherein at least 70% of the cells in the cell population express a cardiac progenitor cell (CPC) associated marker and 1% or less of the cells in the cell population express Octamer Transcription Factor 4 (OCT4) or OCT4/OCT3. The disease may be any disease as disclosed herein.

### 4.18 Swiss-Style

The disclosure also relates to the use of the cell population of the disclosure for the manufacture of a medicament for the treatment or prevention of disease in a subject. The disclosure also relates to the use of a cell population for the manufacture of a medicament for the treatment or prevention of disease in a subject wherein at least 70% of the cells in the cell population express a cardiac progenitor cell (CPC) associated marker and 1% or less of the cells in the cell population express Octamer Transcription Factor 4 (OCT4) or OCT4/OCT3. The disease may be any disease as disclosed herein.

### 5 TERMINOLOGY

### 5.1 AZD6414

AZD6414 cells are derived from hESCs with a differentiation protocol (addition of GSK3 inhibitor at Day 0 and of WNT antagonist at Day 3 of differentiation). The cells are currently harvested at Day 8 of differentiation, followed by depletion of potentially pluripotent stem cells using negative magnetic-activated cell sorting (depletion of cells expressing pluripotency-associated protein TRA-1-60).

The starting material for AZD6414 production is GMP MCB H9 derived from source cell line H9 (WIC-WA09), a human embryonic cell line.

### 5.2 Cell Potency

Pluripotent cells are cells that can self-renew and proliferate while remaining in an undifferentiated state and that can, under the proper conditions, be induced to differentiate into specialized cell types. The term “pluripotent cells,” as used herein, encompass embryonic stem cells and other types of stem cells, including foetal, amnionic, or somatic stem cells. Exemplary human stem cell lines include the H9 human embryonic stem cell line. Additional exemplary stem cell lines include those made available through the National Institutes of Health Human Embryonic Stem Cell Registry and the Howard Hughes Medical Institute HUES collection [11].

Pluripotent stem cells have the potential to differentiate into any of the three germ layers: endoderm (e.g., the stomach linking, gastrointestinal tract, lungs, etc), mesoderm (e.g. muscle, bone, blood, urogenital tissue, etc) or ectoderm (e.g. epidermal tissues and nervous system tissues). The term “pluripotent stem cells,” as used herein, also encompasses “induced pluripotent stem cells”, or “IPSCs”, a type of pluripotent stem cell derived from a non-pluripotent cell. Examples of parent cells include somatic cells that have been reprogrammed to induce a pluripotent, undifferentiated phenotype by various means. Such “IPS” or “IPSC” cells can be created by inducing the expression of certain regulatory genes or by the exogenous application of certain proteins. Methods for the induction of IPS cells are known in the art [12-15].

The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least several, and may express all of the markers SSEA-3, SSEA-4, TRA-1-60, TRA-1-81, TRA-2-49/6E, ALP, SOX2, E-cadherin, UTF-1, OCT4, Rexl, and NANOG.

Multipotent cells can give rise to a limited number of other particular cell types. For example, induced multipotent cells are capable of forming endodermal cells. Additionally, multipotent blood stem cells can differentiate itself into several types of blood cells, including lymphocytes, monocytes, neutrophils, etc. Oligopotent cells are adult stem cells that can differentiate into only a few different cell types. For example, lymphoid or myeloid stem cells are capable of forming cells of either the lymphoid or myeloid lineages, respectively. Unipotent cells form a single cell type. For example, spermatogonia stem cells are only capable of forming sperm cells. Totipotent cells have the ability form entire organism. For example, in mammals, only the zygote and the first cleavage stage blastomeres are totipotent. Differentiated cells include, but are not limited to, multipotent cells, oligopotent cells, unipotent cells, progenitor cells, and terminally differentiated cells. Differentiated cells include, but are not limited to, multipotent cells, oligopotent cells, unipotent cells, progenitor cells, and terminally differentiated cells.

Non-pluripotent cells are not pluripotent cells. Examples of such cells include differentiated cells as well as progenitor cells. Examples of differentiated cells include, but are not limited to, cells from a tissue selected from bone marrow, skin, skeletal muscle, fat tissue and peripheral blood. Exemplary cell types include, but are not limited to, fibroblasts, hepatocytes, myoblasts, neurons, osteoblasts, osteoclasts, and T-cells. The starting cells employed for generating the induced multipotent cells, the endodermal progenitor cells, and the hepatocytes can be non-pluripotent cells.

### 5.3 Cardiomyocyte

The term cardiomyocyte refers to a muscle cell of the heart (e.g., a cardiac muscle cell). A cardiomyocyte will generally express on its cell surface and/or in the cytoplasm one or more cardiac-specific marker. Suitable cardiomyocyte-specific markers include, but are not limited to, cardiac troponin I, cardiac troponin-C, tropomyosin, caveolin-3, GATA-4, myosin heavy chain, myosin light chain-2a, myosin light chain-2v, ryanodine receptor, and atrial natriuretic factor.

### 5.4 Cell Dissociation

Cell dissociation is the process during cell passaging where cells are detached from the tissue culture surface. Dissociated cells are not attached to each other or a cell surface. Dissociated cells may be in suspension. Dissociated cells may be single cells in suspension. Cell passaging of adherent cells involves cell passage. Adherent cells can then be dissociated from the substrate or flask (e.g., by using a protease such trypsin or collagenase), media can be added, optional washing (e.g., by centrifugation) may be performed, and then the cells can be replated or reseeded to one or more culture vessels Other methods of removing non-adherent cells include steps of non-enzymatic treatment (e.g., with EDTA).

### 5.5 Conditions Suitable for Suitable for Cardiac Myogenesis

Methods suitable for differentiating stem cells into cardiac ventricular progenitor cells are described in Foo et al. [6], WO 2016/029122 A1 [7], WO 2017/172086 A1 [8], WO 2018/100433 A1 [9], and WO 2019/038587 A1 [10].

### 5.6 Genes

TABLE 1
Gene identifiers

| Gene | Full name |
|---|---|
| OCT4 (POU5F1) | Octamer-binding transcription factors 4; |
| | (POU class 5 homeobox 1) |
| | Gene ID: 5460 |
| ISL1 | islet1 |
| GSK3 | Glycogen synthase kinase 3 |
| WNT | Wingless-related integration site pathway |
| FGFR3 | Fibroblast growth factor receptor 3 |
| LIFR | Leukemia Inhibitory Factor Receptor |
| TNFSF9 | TNF Superfamily Member 9 |

Expression may refer to gene expression (e.g. as measured by transcript, mRNA analysis or RNA seq) or protein expression (e.g. as measured by immunofluorescence, western blot or flow cytometry).

### 5.7 Harvesting Cells

The process of harvesting cells means isolating the cells or population of cells from the growth or culture media. A population of cells growing in culture may be harvested by dissociating and resuspending the cells.

### 5.8 High Efficiency Culture Assay to Evaluate Stem Cell Contamination in Cell Therapy Products

Undifferentiated hPSCs possess the potential to form teratomas, and thus residual undifferentiated hPSCs are one of the major hazards for the risk of tumour formation from hPSC-derived cell therapy products. Among currently available assays, a highly efficient culture (HEC) assay is reported to be one of the most sensitive for the detection of residual undifferentiated hPSCs [16-18]. Cell therapy product is cultured under conditions optimized for stem cell proliferation for 7 days. Colonies of proliferative undifferentiated stem cells are identified by staining with fluorescent antibodies for undifferentiated stem cells marker proteins. Colonies positive for Oct4 with more than 7 cells (representing 3 divisions in 7 days) is considered a positive colony.

An international validation trial conducted under the HESI CT-TRACS initiative showed that approximately 50% of undifferentiated cells spiked into a differentiated population will be detected as colonies in the high efficiency culture assay [18].

### 5.9 Octamer Transcription Factor 4 (OCT4)

OCT4 (gene POU5F1 (POU class 5 homeobox 1) (Gene ID: 5460, HUGO Gene Nomenclature Committee, HGNC)) (also known as OCT3 or OCT3/4), is a protein that in humans is encoded by the POU5F1 gene. It was originally identified as a DNA-binding protein that activates gene transcription via a cis-element containing an octamer motif. It is expressed in totipotent embryonic stem and germ cells. This protein is critically involved in sustaining self-renewal of undifferentiated embryonic stem cells and pluripotency. OCT4 is not only a master regulator of pluripotency that controls lineage commitment but is also the first and most recognized marker used for the identification of totipotent ES cells. It has been implicated in tumorigenesis of adult germ cells.

### 5.10 Stem Cells

For the avoidance of doubt, the human embryonic cells used in the disclosure can be derived from parthenogenetically activated human oocytes.

The stem cells may be pluripotent. The stem cells may not be totipotent.

### 5.11 Teratomas

Teratomas are tumours containing tissues from all embryonic germ lines (endoderm, ectoderm, and mesoderm).

### 5.12 TRA-1-60 (Podocalyxin)

TRA-1-60 is a cell surface antigen, expressed in human embryonic stem cells, embryonal carcinoma cells and induced pluripotent stem cells (IPS)

### 5.13 Ventricular Progenitor Cells

Ventricular progenitor cells are cells that have the capacity to differentiate into functional, beating ventricular cardiomyocyte tissue when delivered to the ventricular wall of a heart.

### 6 EXAMPLE 1: MATERIALS AND METHODS

### 6.1 Cardiac Progenitor Cell Differentiation Protocol

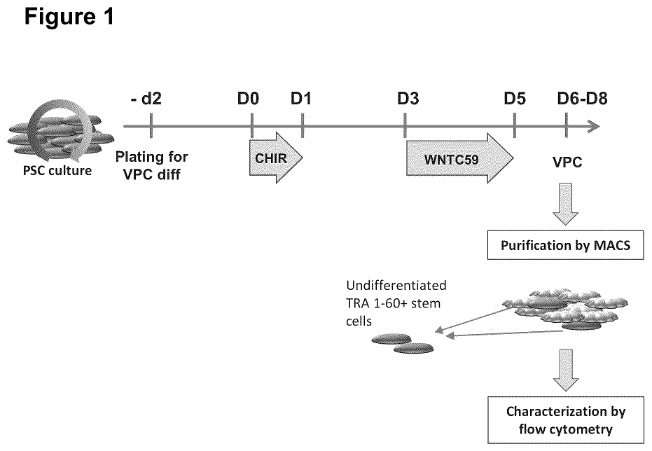

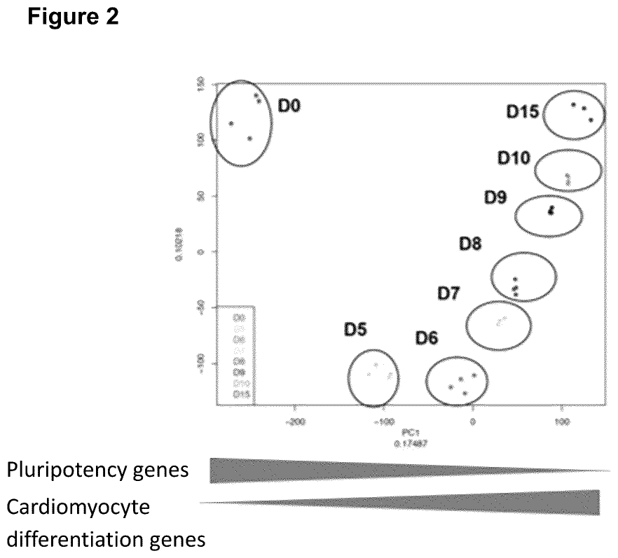

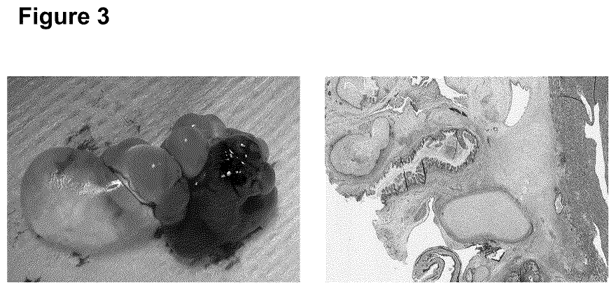

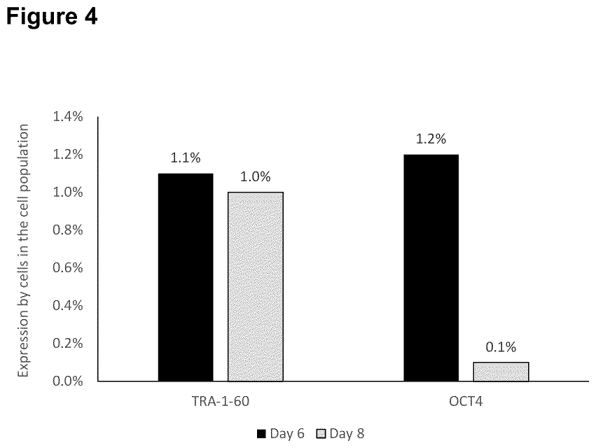

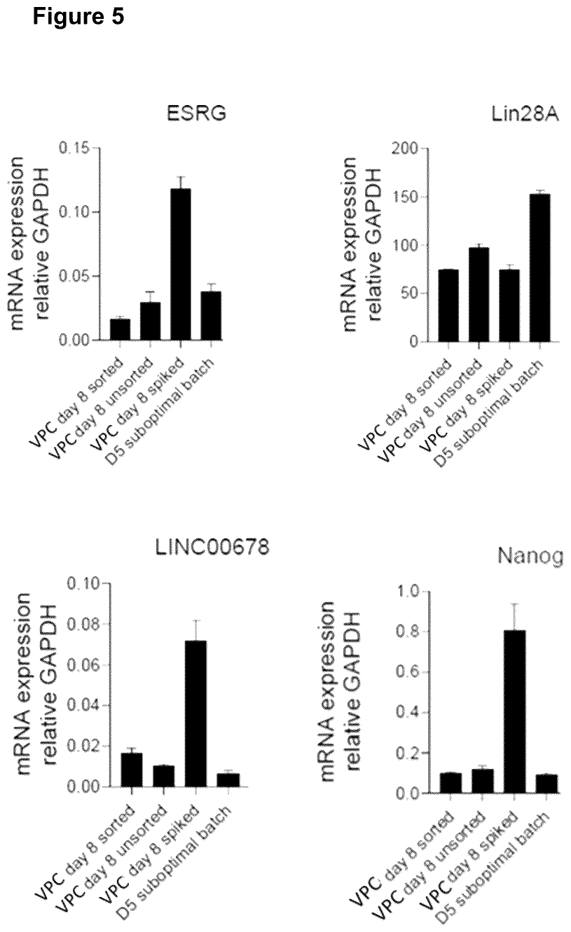

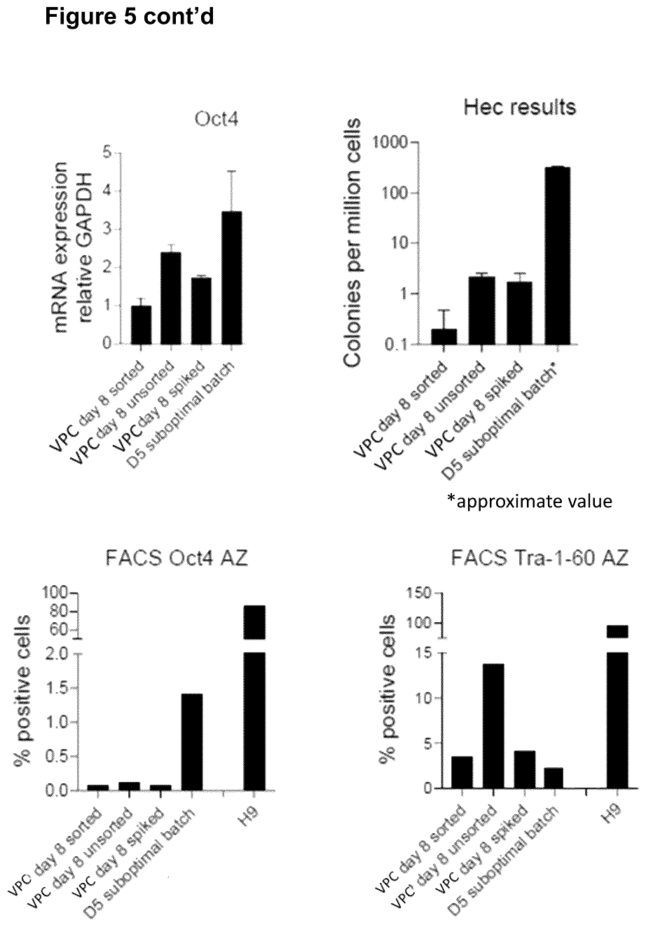

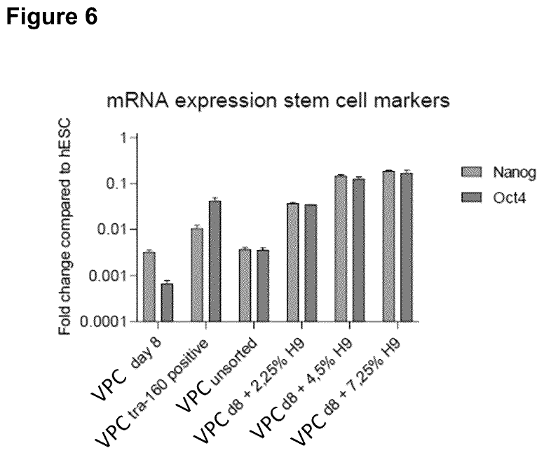

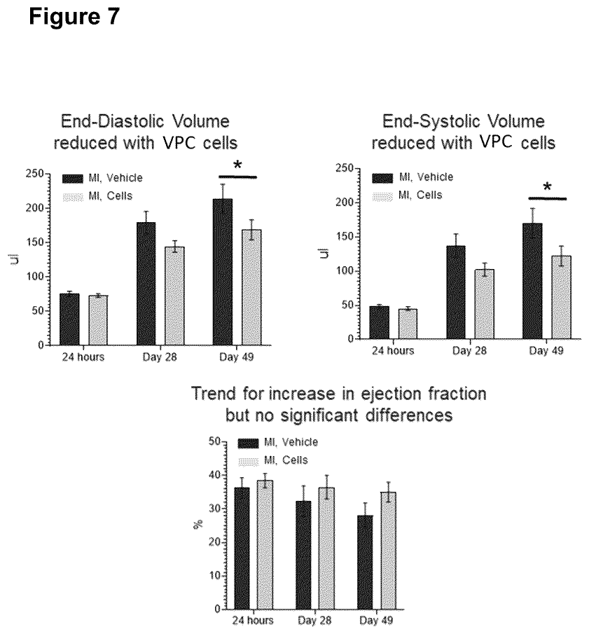

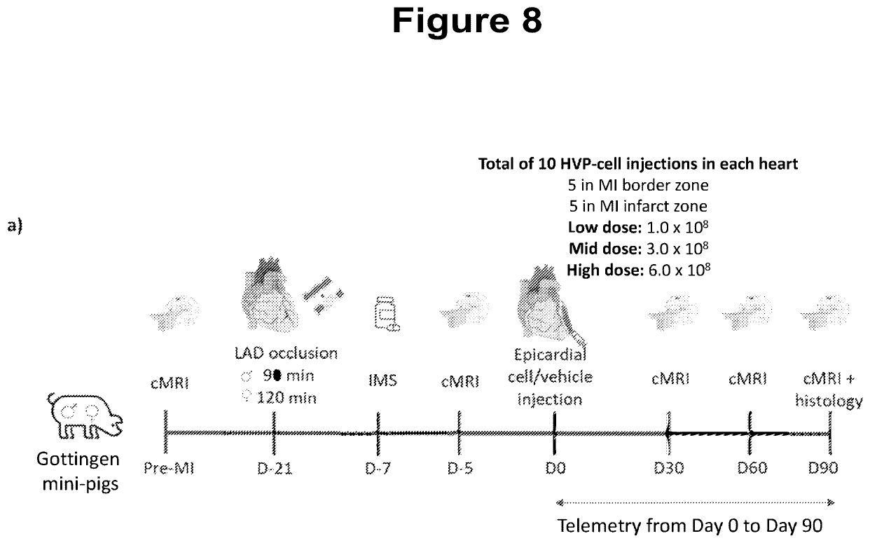

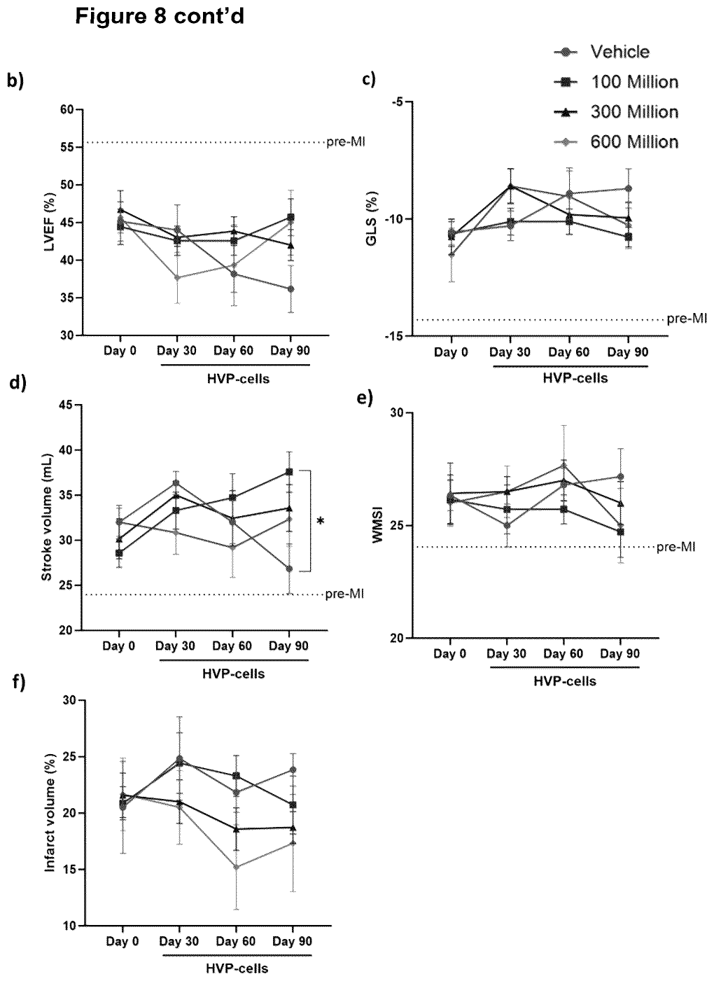

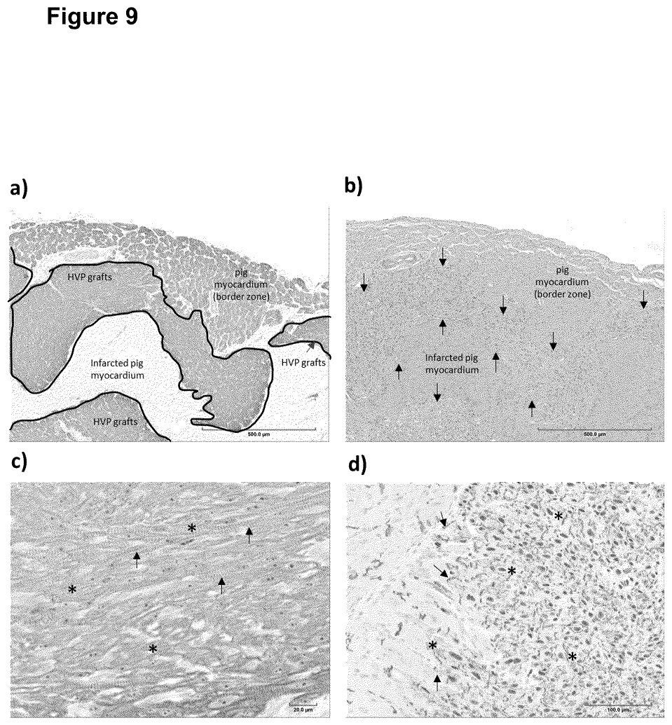

A schematic of the differentiation protocol for cardiac progenitor cells from stem cells is shown in FIG. 1.

### 6.2 VPC Harvesting and TRA-1-60 Magnetic Activated Cell Sorting

VPC harvesting and TRA-1-60 sorting was performed according to standard protocols. Equipment and reagents are provided in Table 2 and Table 3.

TABLE 2
Equipment for cell harvesting & TRA-1-60 magnetic cell sorting

| Equipment | Brand |
|---|---|
| AutoMACS Pro separator | (Miltenyi Biotech) |
| Traceable MicroIR Thermometer | VWR |
| Nucleo ®Counter NC-202 ™ | Chemometech |

TABLE 3
Reagents for cell harvesting TRA-1-60 magnetic cell sorting

| Reagents | Cat. No. | Supplier |
|---|---|---|
| Accutase | A6964 | Merck, Sigma-Aldrich |
| AutoMACS Running buffer | 130-091-221 | Miltenyi Biotech |
| Washing Solution | 130-092-987 | Miltenyi Biotech |
| CryoStor CS10 | 07930 | StemCell Technologies |
| Anti TRA 1-60 Microbeads | 130-100-832 | Miltenyi Biotech |
| human | | |
| AutoMACS columns | 130-021-101 | Miltenyi Biotech |

### 6.2.1 Cell Harvesting

In brief, day 8 ventricular progenitor cells were harvested. To detach the cells, Accutase was added to the adherent cell culture at a concentration of 0.1 mL/cm² and incubated for 2-5 mins at 37° C. Cold AutoMACS running buffer (0.1 mL/cm² was added to culture vessel to dilute the Accutase. The detached cells were resuspended to produce a single cell suspension and transferred to a centrifuge tube. The single cell suspension was then centrifuged at 300×g for 6 minutes. The supernatant was discarded, and the cell pellet resuspended into a single cell suspension in AutoMACS running buffer to a volume corresponding to 0.1 mL/cm² of the collected vessels. The single cell suspension was then centrifuged at 300×g for 6 minutes. The supernatant was discarded, and the cell pellet resuspended into a single cell suspension in AutoMACS running buffer to a volume corresponding to 0.01 mL/cm² of the collected vessels. The cells were counted using NC-202.

### 6.2.2 TRA-1-60 Sorting Cells

The total number of unsorted ventricular progenitor cells was calculated to add the correct amount of anti TRA-1-60-conjugated microbeads/1 mL. The single cell suspension was centrifuged at 300×g for 6 minutes at 4° C. The pellet was resuspended in cold AutoMACS running buffer, 66.7*10⁶ cells/mL (total cells). 667 μL of anti TRA-1-60-conjugated microbeads/1 mL were added and incubated 10 minutes at 4° C. in AutoMACS chill rack on a rotary shaker (in cold room, set 50 rpm). After incubation, 1 ml of AutoMACS running buffer was added per 40×10⁶ in the tube and the cells centrifuged at 300×g for 6 minutes at 4° C. The cells were re-suspended in cold AutoMACS running buffer at 40*10⁶/mL (total cells). The samples were then placed in to the AutoMACS Pro separator. The sorted cell populations may be frozen after sorting.

### 6.3 High Efficiency Culture (HEC) Assay to Evaluate Stem Cell Contamination in Cell Therapy Products

The HEC assay was performed according to standard protocols. In brief, the cell therapy product to evaluated was thawed and/or dissociated. 9 million cells were removed to a 50 ml tube. 9 million cell assays will allow for predictions down to approximately ˜220 cells in a 1 billion dose, assuming 50% colony forming rate. The cells were seeded onto 6 well plates and incubated for 7 days, with media change every 2-3 days, in complete E8 without Rock inhibitor.

The cells were then fixed with PFA and stained with alkaline phosphate stain (Vector, SK-5300) and an antibody stain according to standard protocols. The plates were imaged on ImageXpress micro. Materials used in the HEC assay are shown in Table 4.

TABLE 4
Reagents for HEC assay

| Name | Supplier | Cat no | Working conc | |
|---|---|---|---|---|
| LN521 (iPSCs) | Corning | 354224 | 20 | μg/ml |
| E8 Flex | Thermo Fischer | A2858501 | 1x | |
| Y27632 | Tocris | 1254 | 10 | μM |
| PFA 4% | Thermo Fisher | AAJ19943K2 | 4% | |
| Oct-4 antibody 488 | Abcam | ab208907 | 1:100 dilution | |
| Oct-4 antibody | Abcam | ab200834 | 1:500 dilution | |
| FBS | Thermo Fischer | 26140079 | 10% | |
| PBS | Thermo Fischer | 14190144 | 1x | |
| Triton-X | Invitrogen | HFH10 | 0.10% | |
| Alkaline phosphatase | Vector | SK-5300 | 1x | |
| stain | | | | |
| Tris HCL | Merk | 10812846001 | 100 | mM |

| | | | |
|---|---|---|---|
| Alexa 596 anti-rabbit | Invitrogen | A-11012 | 1:1000 dilution |

### 6.4 Flow Cytometry

Flow cytometry was performed according to standard protocols. Reagents are shown in Table 5.

TABLE 5
Reagents for flow cytometry

| Reagent | Cat. No. | Supplier |
|---|---|---|
| AutoMACS running buffer | 130-091-221 | Miltenyi Biotec |
| BSA Fraction V (7.5%) | 15260-037 | Thermo Fisher Scientific |
| Formaldehyd 4% | 9713.5000 | VWR |
| Live/Dead Fixable Violet | L34964 | Thermo Fisher Scientific |
| Dead Cell Stain kit | | (Invitrogen) |
| PBS (without | 10010-015 | Thermo Fisher Scientific |
| calcium/magnesium) | | |
| Triton X-100 | 11332481001 | Sigma-Aldrich |

Intracellular antibodies and
their Isotype controls

| | | |
|---|---|---|
| Anti-Cardiac Troponin-T | 130-120-403 | Miltenyi Biotec |
| (APC) | | |
| Nanog (PE) | 130-117-377 | Miltenyi Biotec |
| Anti-Oct 3/4 (APC) | 130-117-709 | Miltenyi Biotec |
| Anti-SOX-2 (PE) | 130-121-053 | Miltenyi Biotec |
| Mouse anti-ISL1-1 (PE) | 562547 | BD |
| Mouse IgG1, K Isotype | 554680 | BD |
| Control to ISL1 (PE) | | |
| REA Control antibody (APC) | 130-120-709 | Miltenyi Biotec |
| REA(I) PE, Istotype Control | 130-118-347 | Miltenyi Biotec |
| to SOX-2 (PE) | | |

Surface antibodies and
their Isotype controls

| | | |
|---|---|---|
| Anti-Tra-1-60 (PE) | 130-122-921 | Miltenyi Biotec |
| REA (S) PE, Isotype control | 130-113-438 | Miltenyi Biotec |
| to Tra-1-60 (PE) | | |

### 6.5 RNA-Sequencing

RNA sequencing was performed according to standard protocols. In brief cryopreserved cells were thawed and counted using a standard trypan blue exclusion assay. After assessing cell count and viability, scRNA-sequencing was performed using the Chromium Next GEM Single cell 3′ Reagent Kits v3.1 (10× Genomics). Briefly, cells and reagents were captured using a Chromium Controller, followed by reverse transcription, cDNA amplification and library construction, according to the manufactures recommendations. The libraries were sequenced on a NovaSeq 6000 (Illumina) instrument using 28, 8 and 91 cycles.

### 6.6 RNA Isolation, RT-PCR and RT-qPCR.

RT-PCR was performed according to standard protocols. In brief a cell pellet of approximately 1 million viable cells were lysed using RLT buffer (Qiagen) and either cryopreserved at −80° C. or processed straight away. RNA was isolated from cell lysate by utilizing a column based separation with on column DNA digest (Qiagen, Rneasy mini kit) and RNA concentration determined using a spectrophotometer (Nanodrop). A two-step RT-qPCR was then performed, for cDNA generation typically 1 ug RNA was utilized for reverse transcription into cDNA and subsequential qPCR was performed using 12 ng of cDNA using TaqMan master mix and Taqman RT PCR assays. qPCR was performed by a QuantStudio 7 Flex instrument and analyse using the QuantStudio Software. Reagents: TaqMan™ Fast Advanced Master Mix (2×) Catalogue number: 4444557, High-Capacity cDNA Reverse Transcription kit Catalogue number: 4368814, Instrument Type: QuantStudio™ 7 Flex.

### 6.7 Primers and Probe Sequences

Primers and probes for use in the methods of the disclosure are known in the art. See also Table 6.

TABLE 6
Example primers and probes

| Gene | TagMan assay ID |
|---|---|
| OCT4/POU5F1 | Hs03005111_g1 |
| ISL1 | Hs00158126_m1 |
| NANOG | Hs02387400_g1 |

### 6.8 H9 Cells

The GMP MCB H9 is derived from the source cell line WiCell WA (WiCell Research Institute) 09, also referred to as H9. This material consists of a hESC line first described by Thomson et al. [20], which was chosen based on the criteria that these cells still maintained the developmental potential to form trophoblast and derivatives of all 3 germ layers (the endoderm, ectoderm, and mesoderm) after feeder-less growth. H9 cells have been used for clinical studies [21,22]. In 2009, a current GMP bank of H9 cells was established from cells at passage 27 by Waisman Biomanufacturing (WiCell Certificate of Analysis).

### 7 EXAMPLE 2: DIFFERENTIATION WINDOW FOR CARDIAC PROGENITOR CELLS

### 7.1 Introduction

Foo et al. [6] describes the generation of human pluripotent stem cell (hPSC)-derived ventricular progenitor cells (HVPs). Foo et al, particularly describes a method wherein Day 6 of differentiation of HVPs from embryonic stem cells (ESCs), the HVPs were harvested and enriched by depleting cells positive for a pluripotency marker [6]. Day 6 was selected as the harvest window for the HVPs by Foo et al., based on peak ISL1 expression, and the lack of teratoma formation following engraftment of Day 6 HVPs into a murine kidney, with or without a pre-sorting step to deplete cells of pluripotent cells. Foo et al, particularly identifies day 6 of differentiation as a unique developmental window for HVP engraftment.

### 7.2 Reassessing Cardiac Progenitor Differentiation

The present inventors confirmed that populations differentiating from stem cells towards cardiomyocytes in suitable conditions demonstrated good gene expression change from pluripotent state towards a differentiated cardiomyocyte population (FIG. 2).

However, the present inventors surprisingly found that populations of cardiac progenitors harvested at Day 6 of differentiation from stem cells, as recommended by Foo et al., still expressed unacceptable levels of pluripotency associated genes. Teratomas were also found in the kidneys of mice injected with Day 0, 5, and 6 of unsorted cell populations (Table 7, FIG. 3).

TABLE 7
Teratoma formation by engrafted populations
of cardiac progenitor cells

| Group | Differentiation day | Teratomas | TRA 1-60 (%) |
|---|---|---|---|
| Unsorted cells | 0 | 4/5 | 97.7 |
| | 5 | 5/5 | 45.5 |
| | 6 | 3/5 | 37.4 |
| | 7 | 0/5 | 30.9 |
| | 8 | 0/5 | 13.7 |
| | 9 | 0/5 | 5.1 |
| | 10 | 0/5 | 2.1 |
| | 15 | 0/5 | 0.0 |

Cells generated in T175 cm² flask format; Grafts formed in all conditions; TRA-1-60: undifferentiated stem cell marker. Grafts injected under the kidney capsulae of mice and assessed 2 months post-engranftment.

An alternative differentiation protocol was devised, whereby a population of cardiac progenitor cells was harvested from the cell culture at Day 8. FACS analysis revealed that, at Day 8, the population of cardiac ventricular progenitor cells (VPCs) retained expression of a cardiac progenitor associated marker (ILS1), and had reduced expression of pluripotency markers, including TRA-1-60, OCT4, NANOG and SOX2 (Table 8). OCT4 expressing cells were still present at high levels in Day 6 harvested cells, even after TRA-1-60 sorting (FIG. 4). By contrast, the percentage of cells expressing OCT4 in Day 8 harvested and TRA-1-60 depleted cells was almost undetectable (about 0.1%) (FIG. 4). In other words, in contrast to Day 6 populations, at Day 8 the cells were still in a cardiac progenitor state but had transitioned out of the dangerous pluripotent state.

TABLE 8
Gene expression analysis

| | |
|---|---|
| Day 6 | Day 8 |

| Marker | Pre-sort | Post-sort | Pre-sort | Post-sort |
|---|---|---|---|---|
| ISL1 | 93% | 93% | 86% | 91% |
| TRA-1-60 | 39% | 1.1% | 15% | 1.0% |
| OCT4 | 1.4% | 1.2% | 0.3% | 0.1% |
| NANOG | 0.5% | 0.2% | 0.1% | 0.1% |
| SOX2 | 3.4% | 2.5% | 2.3% | 2.4% |

ISL1: cardiac progenitor call marker; TRA-1-60, OCT4, NANOG, SOX2; undifferentiated stem cell marker. % expression assess by FACS

### 7.3 Detecting Residual Pluripotency in Cell Cultures

To better understand the contamination of undifferentiated cells in the cell populations harvested from Day 6 cells, further analysis of markers of undifferentiated stem cells was conducted using a high efficiency culture (HEC) assay. The HEC assay is considered one of the most sensitive methods of detection of residual undifferentiated pluripotent stem cells in a cell population for use in cell therapy [23]. The HEC assay is suitable for detecting low numbers of undifferentiated stem cells contamination within a differentiated cell population. The HEC assay can detect colonies formed from PSCs using a highly efficient culture system, which favours the growth of PSCs, and the limit of detection (LOD) has been reported to be 0.0002% [23,24].

Using the HEC assay, cell populations pre- and post-TRA-1-60 depletion were assessed for expression of pluripotency associated genes (OCT4 and NANOG). As a positive control, a population of Day 8 cells were spiked with pluripotent (undifferentiated) stem cells (H9) (Table 9).

TABLE 9
Pluripotent gene expression in harvested cardiac progenitor populations

| | | | HEC assay | | | | |
|---|---|---|---|---|---|---|---|
| | | No. of | | | OCT4⁺ | | |
| | | cells | OCT4⁺ | | colonies/ | | |
| | TRA-1-60 | analyzed | NANOG⁺ | OCT4⁺ | million | FACS (% cells) | |
| Day of harvest | depleted | (million) | colonies | colonies | cells | OCT4⁺ | TRA-1-60⁺ |
| Day 8 | Yes | 10 | 2 | 2 | 0.2 | 0.07 | 3.56 |
| Day 8 | No | 8 | 15 | 17 | 2.13 | 0.12 | 13.7 |
| Day 8, with 0.0001% | Yes | 10 | 16 | 17 | 1.7 | 0.07 | 4.12 |
| H9 spiking | | | | | | | |
| Day 5 | Yes | 10 | ~4278 | ~6111 | ~611.1 | 1.42 | 2.32 |

Inclusion criteria: minimum of 8 cells per colony, representing 3 divisions of a single cell in 7 days. % OCT4+ cells measured by FACS; All cell populations derived from H9 cells. ~D5 cells have estimated values as colonies were too close to determine individual colonies.

The results of the HEC assay demonstrated that Day 8 colonies of harvested populations of cardiac progenitor cells depleted of TRA-1-60 cells had very low levels of residual undifferentiated pluripotent stem cells, as measured by OCT4 and/or NANOG expression (FIG. 5).

Embryonic stem cell-related gene (ESRG), and long intergenic non-protein coding RNA 678 (LINC00678) and Lin28A have also been identified as markers of undifferentiated stem cells in a differentiated cell therapy product [25]. The inventors assessed whether expression of these genes was predictive of colony formation in the HEC assay. The mRNA levels of ESRG, NANOG and LINC00678 (as measured by qPCR) were able to detect spiking of the cultures with undifferentiated stem cells (0.0001% H9 spiking), but were not predictive of colony formation in the HEC assay (FIG. 5). In other words, ESRG, NANOG or LINC00678 qPCR was not as sensitive an assay for residual pluripotency as the HEC assay. By contrast, OCT4 mRNA levels as measured by qPCR was predictive of colony formation in the HEC assay (FIG. 5). Lin28A qPCR was unable to distinguish between the sorted and unsorted cardiac ventricular progenitor cells.

Surprisingly, TRA-1-60 FACS of the sorted and unsorted cell populations was not predictive of HEC colony formation. In other words, TRA-1-60 FACS was not as sensitive an assay for residual pluripotency as the HEC assay (FIG. 5). Particularly, TRA-1-60 FACS of sorted Day 5 harvested cells was not predictive of HEC colony forming potential of this population. By contrast, OCT4 FACS values were predictive of HEC colony formation (FIG. 5, Table 9).

In a further study, OCT4 expression (as measured by qPCR and FACs) was shown to increase with dose dependent spiking of harvested Day 8 cardiac progenitor cell cultures (Table 10), demonstrating the sensitivity of the qPCR and FACS assays.

TABLE 10
Undifferentiated marker expression in spiked Day
8 harvested differentiated cell populations

| Sample | TRA-1-60 | OCT4 | Viability |
|---|---|---|---|
| VPC D 8 Sorted | 2.40% | 0.07% | 76.7% |
| VPC D 8 TRA-1-60 positive | 8.12% | 3.06% | 81.3% |
| VPC D 8 unsorted | 9.28% | 0.22% | 81.5% |
| Undifferentiated | 96.0% | 84.7% | 95.3% |
| 2.25% H9 spiked in VPC D 8 sorted | 4.27% | 1.82% | 77.3% |
| 4.5% H9 spiked in VPC D 8 sorted | 10.8% | 4.72% | 89.4% |
| 7.5% H9 spiked in VPC D 8 sorted | 12.2% | 6.68% | 90.1% |

VPC: cardiac ventricular progenitor cells

### 7.4 Summary

In summary, measuring OCT4 levels by both mRNA and FACS was predictive of HEC assay outcome, where HEC colony formation is considered the most sensitive assay for assessing residual pluripotency in a cell therapy product. Measuring OCT4 levels by mRNA or FACS in a cell therapy product is therefore considered a suitable method for assessing the risk of teratoma formation in a population of harvested cardiac progenitor cells. Furthermore, the HEC assay validated OCT4 FACS and qPCR analysis demonstrated the superiority of Day 8 versus Day 6 populations of cardiac progenitor cells (Table 8, FIG. 5).

### 8 EXAMPLE 3: AZD6414 MANUFACTURE

Based on the new differentiation protocol described in EXAMPLE 2, the inventors devised a method for producing a cell therapy product (AZD6414) comprising population of cardiac ventricular progenitors for use in the treatment of heart disease.

The starting material for AZD6414 was produced in GMP MOB H9 derived from source cell line H9 (WIC-WA09), a human embryonic cell line. Manufacture started with the thawing of H9 cells from MCB followed by cell culture (expansion and differentiation) in 2D-adherent monolayer culture using tissue culture flasks. Differentiated cultures, containing cardiac progenitor cells, are then harvested, purified, and cryopreserved.

Expansion started with the thawing of frozen cells from the MCB. Cells are then washed and expanded in 2D-monolayer (tissue culture flasks). In-process monitoring during expansion includes cell viability and viable cell count as well as microscopic inspection to determine hESC morphology and cell confluency according to a standard operating procedure. Medium exchange was performed daily.

Differentiation phase started with seeding the expanded cells in medium at controlled cell density. Medium exchange was performed daily, and cells are monitored (morphology and confluency) according to standard operating procedures.

On Day 2 after seeding, cardiac differentiation was initiated as follows:
Day 0 GSK3 inhibition: Growth medium (mTeSR1) is replaced with differentiation medium (RPMI 1640/B27 minus insulin with CHIR-98014) (cell permeable GSK3 inhibitor).
Day 1 GSK3 inhibition removal: Differentiation medium is replaced with RPMI 1640/B27 minus insulin.
Day 3 WNT inhibition: Medium is replaced with RPMI 1640/B27 minus insulin with Wnt-C59.
Day 5 WNT inhibition removal: Medium is replaced with RPMI 1640/B27 minus insulin.
Day 7 Medium is replaced with RPMI 1640/B27 minus insulin.
Day 8 Cell harvest, purification of cardiac progenitor cells (AZD6414), formulation, and cryopreservation.

Purification phase started with the harvest of differentiated cells, subsequent washing, and incubation with anti-TRA-1-60 immuno-magnetic beads. Purification was achieved by depletion of cells expressing the pluripotency-associated protein TRA-1-60, using magnetic-activated cell sorting.

Purified cells were then concentrated and the medium from the purification phase is removed. Next, the concentrated cells were formulated in freezing medium. The drug product was then filled into cryovials, visually inspected, and labelled, before being transferred to a controlled rate freezer for cryopreservation. Once cryopreservation was complete, the frozen drug product vials were moved into vapor-phase liquid nitrogen for long-term storage.

### 9 EXAMPLE 4: IN VIVO TOXICOLOGY OF AZD6414

Cardiac delivery of AZD6414 was assessed in a toxicology programme comprised of rodent studies, large animal studies, and in vitro assessments. The key safety concerns for AZD6414 are related to teratoma and teratocarcinoma formation.

### 9.1 Assessment of Risk of Teratoma and Teratocarcinoma

As described in Example 2, a key risk connected with pluripotent stem cell-derived cells is teratoma formation (tumours containing tissues from all embryonic germ lines [endoderm, ectoderm, and mesoderm]). The H9 hESC line was used as source material in manufacturing of AZD6414 and the manufacturing process was designed to remove residual pluripotent cells after differentiation into cardiac progenitor cells using magnetic-activated cell sorting with negative sorting (TRA-1-60 marker). In addition, residual levels of pluripotent cells were controlled at drug product release using markers associated with pluripotency (e.g. OCT3/4).

A combined analysis showed that in vitro analysis provides greater sensitivity and translatable data to define levels of pluripotent stem cell contamination than the in vivo study (Table 11). As described in EXAMPLE 2, the in vitro HEC assay is considered to be highly sensitive and more likely to identify potential pluripotent cell contaminants than traditional in vivo investigations [23]. The HEC assay has been reported to be able to detect iPSCs spiked into primary human mesenchymal stem cells or human neurons at the ratio of 0.001% to 0.01% formed colonies. This is a higher sensitivity than is likely to be achieved in vivo.

TABLE 11
Results of In Vitro and In Vivo Investigations
for Potential Teratoma and Teratocarcinoma

| | Test | | | |
|---|---|---|---|---|
| | In vitro | | | |
| | | Soft agar | In vivo | |
| | HEC | assay | Mouse | Minipig |
| | Colonies/ | Colonies/ | Animals with | Animals with |
| Sample | million cells | million cells | teratoma | teratoma |
| AZD6414 | 0 | 0 | 0/8 | 0/7 |
| AZD6414 + 2.25% spike hESC | TNC | NA | 0/5 | NA |
| AZD6414 + 4.5% spike hESC | TNC | NA | 0/5 | NA |
| AZD6414 + 7.5% spike hESC | TNC | NA | 0/5 | NA |
| hESC | TNC | 0 | 5/5 | NA |

hESC = human embryonic stem cells; HEC = high-efficiency culture; NA = not applicable; SC = subcutaneous; TNC = too numerous to count.

### 10 EXAMPLE 5: AZD6114 STUDY IN MOUSE HEART POST-MI (STUDY BS002651-08)

To investigate the effect of AZD6414 derived from H9 on cardiac function in rodents, immunocompromised mice (NOD/SCID gamma) were subjected to MI by permanent occlusion of LAD coronary artery. Two million AZD6414 cells were injected directly into the border zone of the infarcted heart (2 injection sites) and cardiac function was evaluated with echocardiography at Day 1, 28, and 49. AZD6414 attenuated adverse remodelling (end-diastolic volume and end-systolic volume) of the left ventricle, and tended to improve LVEF 49 days after cell injection (FIG. 7). Immunohistological analysis for the human nucleoli showed small clusters of injected cells in the myocardium at Day 49. No evidence was observed of teratoma or teratocarcinoma formation due to AZD6414 derived from H9

### 11 EXAMPLE 6: GLP TOXICOLOGY STUDY

### 6.1 Materials and Methods

6.1.1 Subcutaneous injection of HVP-cells in NSG mice. Five groups of NSG mice (Strain: NOD.Cg-Prkdo^{scid} II2rg^{tm1Wjl}/SzJ, from the Jackson Lab, USA), were administered by subcutaneous bolus injection of placebo (15 animals), HVP-cells only (15 animals), HVP-cells with undifferentiated human embryonic stem cells (at 0.3% or 1%, 7 animals), or Pluripotent Cells (positive control, 5 animals), in diluent control (Matrigel) once on Day 1. The target dose levels for HVP-cells with or without impurities was 20 million cells per animal at a volume of 0.2 mL. Mice were sacrificed after 6 months or earlier due to large tumour growth (positive control animals only). Tissues were examined microscopically for evidence of toxicity and tumourigenicity of HVP-cells.

### 6.1.2 Porcine Experiments for GLP Toxicology/Efficacy Study.

6.1.2.1 Myocardial infarction and immunosuppression in pigs. Male and female Gottingen minipigs (25-30 kg) were from Sinclair Research Center, USA. Following anaesthesia, a small incision was made over the carotid artery and jugular vein, a small opening was made in the artery and a sheath was introduced. Heparin (250-350 IUkg⁻¹) was administered to maintain an activated clotting time twice the baseline activated clotting time level. A guide catheter was advanced into the ostium of the LAD. A balloon catheter was introduced by advancing it through the guide catheter to the LAD and placed below the first diagonal branch of the LAD and inflated to occlude the artery for 90 min in males and 120 minutes in female. It was then deflated to re-perfuse the ischaemic area. All handling and treatments were performed according to the appropriate animal welfare standards and in compliance with regulatory guidelines (IACUC 1974-061) in Charles River, Mattawan (MI, USA). Two weeks following the myocardial infarction procedure, a predose cardiac MRI was performed between Days −5 to −1 to assess left ventricular ejection fraction (LVEF). Animals considered to be suitable for assignment to the study were selected for study based upon survival of the infarct and meeting the enrollment criteria of a LVEF≤45% and/or a reduction in LVEF by at least 15% from baseline values. Animals which met enrollment criteria were randomized into vehicle or HVP-cells (low-dose, 1.0×10⁸ cells; mid-dose, 3.0×10⁸ cells; high-dose, 6.0×10⁸ cells) groups. For immunosuppression, a combination of cyclosporin A, methylprednisolone and abatacept was used (REF, Romagnuolo et al. Stem Cell Reports 12, 967-981; Nat Cell Biol paper). From D-6 to D84, cyclosporin A (20-30 mg kg⁻¹) was given orally twice a day, except for D1. On the morning of D1, cyclosporin A dose was 10 mg kg⁻¹ intravenous infusion. From D-1, methylprednisolone (128 mg d⁻¹) was given until D3, and subsequently tapered to 80 mg d⁻¹ (D4 to D6): 48 mg d⁻¹ (D7 to D9) and 24 mg d⁻¹ from D10 for the remainder of the study. Abatacept (12.5 mg kg⁻¹) was given as 30 min intravenous infusion every second week from D-1. Blood samples for monitoring cyclosporin A levels were collected, ranging from 100 μg l⁻¹ to 1,000 μg l⁻¹, except immediately after the cyclosporin A infusion.

6.1.2.2 Epicardial transplantation of HVP-cells into pig hearts after MI. Three weeks after MI, an incision was made over the femoral artery and vein under anaesthesia and an opening in the artery and a sheath was introduced to monitor blood pressure. A midline incision was made over the sternum, and the skin and underlying musculature was retracted. The pericardium was incised, and sutures were passed through and attached to the chest wall to form a sling. Glass beads were used to indicate injection locations. Vehicle or HVP-cells (n=6 vehicle, n=7 HVP-cells-low, n=7, HVP-cells-mid and n=6 HVP-cells-high) were injected 300 μl per site, using a 30 G needle, in the LV myocardium: five injections in the border zone and five injections in the infarcted area. After final injection, the heart was returned to the pericardial sling and sutures were removed. Pigs were maintained and sacrificed after 8 days or 90 days. Tissue sections from each injection site were stained for haematoxlin and eosin, examined microscopically by a board-certified pathologist and the presence of human cells (confirmed by anti-human nucleoli immunostaining) and any other histopathological changes, including the presence of teratoma were noted. All HVP-derived grafts were negative for teratoma at 3 months. Lung, liver, heart, kidney, spleen, brain, thyroid, adrenal glands, pituitary, prostate and lymph nodes were collected. Human haemoglobin B was not detected by dd-PCR with probe HS00758889_s1 HBB FAM and no signs of HVP-derived cells outside the heart were present.

6.1.2.3 Cardiac magnetic resonance imaging. cMRI was performed under anaesthesia, using a 1.5 T MRI scanner (Philips Intera platform R12 software), pre-MI surgery, Day −7 (after MI and before treatment), D30, D60 and D90 post HVP-cells injection. Short- and long-axis images with a 1 cm interval between slices were obtained. Data were analysed with Circle Cardiovascular Imaging cvi42 (V5.12). Three-dimensional (3D) volumes were calculated as the sum of (area×(slice thickness+distance between slices)) for all short-axis slices. Ejection fraction was calculated as 100× (end diastolic volume−end systolic volume)/end diastolic volume. Late gadolinium enhancement MRI was used to quantify infarct size as percentage volume (%). Global longitudinal strain (GLS) was also analysed. Animals were killed after 12 weeks. Hearts were removed, perfused with lactated Ringer's solution. LV containing all the injection sites was fixed in formalin for 48±12 h, then transferred to 70% ethanol and paraffin embedded.

6.1.3 Immunohistochemistry of HVP-cells. Paraffin-embedded hearts were sectioned into 4 μm slices. Immunohistochemistry methods and protocols were set up on an automated Ventana Discovery Ultra autostainer (V12.5.4; Roche). Immunohistochemistry for detection of desired epitopes (Supplementary Table 1) was carried out according to the manufacturer's recommendation and all reagents except antibodies were Ventana products (Roche). Antigen retrieval was done before primary antibody was added, followed by antibody block and secondary anti-rabbit reagent or anti-mouse horseradish peroxidase, purple teal or DAB chromogenic detection in single or double staining. The slides were digitized using a Aperio XT whole slide scanner and Aperio ImageScope software (V12.3.3.5048).

6.1.4 Statistics. Statistical analyses were performed using GraphPad Prism (V10). Data are presented as mean #s.e.m. unless otherwise indicated. Three groups or repeated measures were analysed using Two Away ANOVA Mixed-effects followed by Dunnett's multiple comparison test. The statistical details for each experiment are also provided in the figure legends. A power calculation was performed with historic in-house data and data distribution determined by QQ plot analysis to determine the sample size. For all other experiments, no statistical methods were used to pre-determine sample size. Data distribution was assumed to be normal and individual data points are presented in all graphs. In the porcine model, animals were evaluated on the basis of ejection fraction after MI, and subsequently randomized to vehicle- or HVP-treatment groups. cMRI was analysed blinded by two independent cardiologists. Ex vivo experiments were randomly assigned to experimental and control groups. Data collection was not performed blinded owing to the experimental conditions.

### 6.2 Results.

6.2.1 Strong efficacy signals confirmed in a 3-month GLP study in pigs with myocardial infarction. In a rigorously conducted GLP study involving pigs with myocardial infarction, we observed robust efficacy signals. Our previous work demonstrated that a dose of 1 billion HVP cells led to successful engraftment in the host myocardium, resulting in reduced infarct volume and attenuation of progressive decline in cardiac function in a pig model of chronic ischemic cardiomyopathy [26]. In this current study, we utilized the same model, inducing myocardial infarction through occlusion of the left anterior descending artery (LAD) in male and female pigs, with the incorporation of lower cell doses. HVP cells at three varied doses, along with a control group receiving a vehicle, were injected into the border zone of the infarcted area and the scar area. Immunosuppressant treatment was initiated 6 days prior to cell delivery and maintained throughout the 3-month study period. Subsequently, a total of 26 pigs underwent cMRI to assess LV function prior to cell injection and at one, two-, and three-months post cell transplantation (FIG. 8a). Before cell transplantation, all groups experienced reduced LV function, with EF averaging 44.85% (Vehicle, 45.00%; HVP-cells-low, 43.88%; HVP-cells-mid, 45.40%; HVP-cells-high, 45.13%). Over the subsequent 3 months, LVEF further deteriorated significantly by approximately-20% in the vehicle-treated group, while the treated groups either mitigated the decline in LVEF or exhibited improvement compared to LVEF before cell transplantation (HVP-cells-low, 4%; HVP-cells-mid, −8%; HVP-cells-high, 0%). Notably, no significant differences were observed between the 100, 300, and 600 million cell dose groups at the 3-month mark following cell transplantation. During the first month, a decline in LVEF was noted in the dosed animals, likely attributed to transient engraftment arrhythmias observed during this period (see next section) (FIG. 8b). These trends in LVEF were mirrored by global longitudinal strain (GLS) (FIG. 8c) and stroke volume (SV) (FIG. 8d) improvements in the dosed animals. Additionally, wall motion score index (WMSI) (FIG. 8e) and infarct volume (FIG. 8f).

This comprehensive assessment of cardiac function and infarct volume over the 3-month period offers compelling evidence of the potential therapeutic impact of HVP cell therapy in the context of chronic ischemic cardiomyopathy, addressing the complexities of myocardial infarction and its repercussions on cardiac function.

6.2.2 Evidence of human cardiac grafts in pig myocardium and absence of teratoma or other neoplastic changes. Histological examination of pig heart tissue, conducted at 8 days and at 3 months post cell transplantation, revealed substantial grafts of human cells within the porcine myocardium, positively expressing ventricular myosin light chain (MLC2v; FIG. 9). In addition, anti-N-cadherin immunostaining of transplanted cells provided evidence of formation of intercalated disc-like structures and integration of human cardiomyocytes within the host pig myocardium. For animals terminated at 3 months, myocardial infarction was induced prior to cell implantation. In these animals, grafts of the human cells were observed in the border and scar areas of the infarcted tissue. The majority of pigs sacrificed at 3-months exhibited minimal to no inflammatory response in the heart at the cell injection sites, which serves as a promising indicator of the efficacy of our immunosuppression regimen (FIG. 9). There was no evidence of teratoma formation or other neoplastic cells following HVP-cells delivery in any pig tissue after 8 days or 3 months nor subcutaneously at the site of injection in NOD mice after 6 months. Furthermore, our analysis did not reveal the presence of human cells in any pig organs other than the heart, and there were no indications of systemic toxicity or neoplastic effects in either cardiac or non-cardiac tissues in either mouse or pig (data not shown). These findings underscore the success of HVP cell transplantation approach, demonstrating not only the integration of human cardiomyocytes within the host myocardium but also the absence of any adverse systemic effects, and further supporting the safety and potential therapeutic value of our novel cell therapy strategy.

### REFERENCES

All publications mentioned in the specification are herein incorporated by reference in their entirety.
[1] A. P. Ambrosy et al., J Am Coll Cardiol 63, 1123 (2014).
[2] T. A. McDonagh et al., Eur Heart J 42, 3599 (2021).
[3] M. A. Pfeffer, A. M. Shah, and B. A. Borlaug, Circ Res 124, 1598 (2019).
[4] G. Savarese and L. H. Lund, Card Fail Rev 3, 7 (2017).
[5] J. A. Iwaz et al., Drugs 76, 187 (2016).
[6] K. S. Foo et al., Mol Ther 26, 1644 (2018).
[7] K. R. Chien and X. Lian, WO2016029122A1 (25 Feb. 2016).
[8] C. Y. LEUNG et al., WO2017172086A1 (5 Oct. 2017).
[9] K. R. CHIEN et al., WO2018100433A1 (7 Jun. 2018).
[10] K. R. Chien, J. Clarke, and C. Y. LEUNG, WO2019038587A1 (28 Feb. 2019).
[11] C. A. Cowan et al., N Engl J Med 350, 1353 (2004).
[12] W. Zhou and C. R. Freed, Stem Cells 27, 2667 (2009).
[13] D. Huangfu et al., Nat Biotechnol 26, 795 (2008).
[14] K. Woltjen et al., Nature 458, 766 (2009).
[15] H. Zhou et al., Cell Stem Cell 4, 381 (2009)
[16] T. Kuroda et al., PLOS ONE 7, e37342 (2012).
[17] S. Kusakawa et al., Sci Rep 5, 17892 (2015).
[18] A. S. Lee et al., Cell Cycle 8, 2608 (2009).
[19] W. M. Schopperle and W. C. DeWolf, STEM CELLS 25, 723 (2007).
[20] J. A. Thomson et al., Science 282, 1145 (1998).
[21] A. H. Kashani et al., Ophthalmol Retina 4, 264 (2020).
[22] J. Piao et al., Cell Stem Cell 28, 217 (2021).
[23] K. Tano et al., PLOS One 9, e110496 (2014).
[24] K. Masumoto et al., Sci Rep 12, 10351 (2022).
[25] M. Lemmens et al., Cytotherapy 25, 59 (2023).
[26] C. Poch et al., Nat Cell Biol 24 (5), 659-671 (2022).

1-99. (canceled)

100. A cell population, wherein:
a) at least 70% of the cells in the cell population express a cardiac progenitor cell (CPC)-associated marker, and
b) 1% or less of the cells in the cell population express Octamer Transcription Factor 4 (OCT4).

101. The cell population of claim 100, comprising dissociated cells, dissociated single cells, or cells in suspension.

102. The cell population of claim 100, wherein the cell population is committed to the cardiac lineage.

103. The cell population of claim 100, wherein the cell population is committed to the ventricular cardiac lineage.

104. The cell population of claim 100, wherein less than 1% of the cells in the cell population express OCT4.

105. The cell population of claim 100, wherein OCT4 expression in the cell population is below the limit of detection as measured by flow cytometry, microarray, RNA sequencing, and/or quantitative polymerase chain reaction (PCR).

106. The cell population of claim 100, wherein:
a) the cell population produces less than one OCT4+ colony per million cells, as measured by high efficiency culture (HEC) assay, or
b) the cells in the cell population do not express OCT4 as measured by Western blot.

107. The cell population of claim 100, wherein 1.5% or less of the cells in the population express T-cell receptor alpha locus 1-60 (TRA-1-60).

108. The cell population of claim 100, wherein 1% or less of the cells in the population express NANOG.

109. The cell population of claim 100, wherein 3% or less of the cells in the population express SRY-box transcription factor 2 (SOX2).

110. The cell population of claim 100, wherein at least 70% of the cells in the cell population express Leukemia Inhibitory Factor Receptor (LIFR) and Islet-1 (ISL1).

111. The cell population of claim 100, wherein at least 75% of the cells in the cell population express a CPC-associated marker.

112. The cell population of claim 100, wherein the CPC-associated marker is Islet-1 (ISL1).

113. The cell population of claim 100, wherein the percentage of cells expressing the CPC-associated marker is measurable or determined by flow cytometry, single-cell RNA sequencing, or immunofluorescence.

114. The cell population of claim 100, comprising cells expressing ventricular progenitor-associated markers.

115. The cell population of claim 100, comprising:
a) cells expressing Islet-1 (ISL1), Platelet-derived growth factor receptor A (PDGFRA), T-box transcription factor 1 (TBX1), Heart and neural crest-derived transcript-1 (HAND1), T-box transcription factor 5 (TBX5), and/or SWI/SNF-related matrix-associated actin-dependent regulator of chromatin subfamily D member 3 (SMARCD3);
b) cells expressing Jagged−1 (JAG1), Frizzled−4 (FZD4), Fibroblast growth factor receptor 3 (FGFR3), Leukemia Inhibitory Factor Receptor (LIFR), and/or TNF Superfamily Member 9 (TNFSF9), or
c) cells expressing LIFR and ISL1.

116. The cell population of claim 100, comprising human cells.

117. The cell population of claim 100, wherein ≤5% of the cells in the cell population are stem cells.

118. The cell population of claim 117, wherein the stem cells are pluripotent stem cells.

119. A method for producing the cell population of claim 100, the method comprising harvesting cells from a cell culture, wherein the harvested cells are derived from stem cells cultured for 8 to 12 days in conditions suitable for cardiac myogenesis.

120. The method of claim 119, wherein the conditions suitable for cardiac myogenesis comprise:
a) activating Wnt/Wnt/β-catenin signalling in the culture at day 0, and
b) inhibiting Wnt/β-catenin signalling in the culture on days 3 to 5 to generate a population of cardiac ventricular progenitor cells (VPCs).

121. The method of claim 120, wherein Wnt/Wnt/β-catenin signalling is activated by a GSK3 inhibitor.

122. The method of claim 120, wherein Wnt/β-catenin signalling is inhibited by a Wnt inhibitor.

123. The method of claim 120, wherein the method comprises depleting the harvested cells of pluripotent cells to produce a purified population of ventricular progenitor cells (VPCs).

124. The method of claim 123, wherein depleting the harvested cells of pluripotent cells comprises depleting the harvested cells of cells expressing a marker of pluripotency.

125. The method of claim 124, wherein the marker of pluripotency is T-cell receptor alpha locus 1-60 (TRA-1-60).

126. The method of claim 120, wherein the stem cells are induced pluripotent cells or embryonic stem cells.

127. A pharmaceutical composition comprising the cell population of claim 100, and a pharmaceutical excipient.

128. A method for treating or preventing a disease, the method comprising administering to a subject an effective amount of the pharmaceutical composition of claim 127.

129. The method of claim 128, wherein the cell population comprises cells derived from a cell or cells that were not isolated from the subject.

130. The method of claim 129, wherein the cells of the cell population are allogeneic to the subject.

131. The method of claim 128, wherein the cell population comprises cells derived from a cell or cells isolated from the subject.

132. The method of claim 128, wherein the disease is associated with loss of myocardium and/or fibrotic scaring, or is heart failure, chronic ischemic cardiomyopathy, or myocardial infarction.

133. The method of claim 128, wherein the subject is receiving immunosuppressive therapy.
